(19) United States
(12) Patent Application Publication
(10) Pub. No.: US 2006/0166974 A1
(43) Pub. Date: Jul. 27, 2006

(54) Azabenzoxazoles for the treatment of CNS disorders

(75) Inventors: Bruce N. Rogers, Mystic, CT (US); Allen J. Duplantier, Ledyard, CT (US); Christopher J. O'Donnell, Mystic, CT (US); Lei Zhang, Groton, CT (US)

Correspondence Address: PFIZER INC, 150 EAST 42ND STREET, 5TH FLOOR - STOP 49, NEW YORK, NY, 10017-5612, US

(73) Assignee: Pfizer Inc.

(21) Appl. No.: 11/271,245

(22) Filed: Nov. 11, 2005

Related U.S. Application Data
(60) Provisional application No. 60/651,463, filed on Nov. 15, 2004.

(51) Int. Cl. A61K 31/551; C07D 487/04

(52) U.S. Cl. 514/221; 540/556

(57) ABSTRACT

The present invention relates to α7 nicotinic receptor agonists of formula I as described herein and to a method for treating disorders of the Central Nervous System (CNS) such as cognitive deficits in schizophrenia, by administering to a mammal an α7 nicotinic receptor agonist of formula I as shown herein.

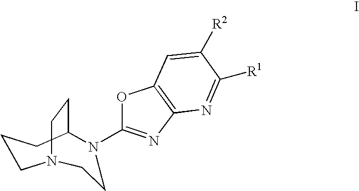

### BACKGROUND OF THE INVENTION

The present invention relates to α7 nicotinic receptor agonists and to a method for treating disorders of the Central Nervous System (CNS) and other disorders in a mammal, including a human, by administering to the mammal an α7 nicotinic receptor agonist. It also relates to pharmaceutical compositions containing a pharmaceutically acceptable carrier and a CNS-penetrant α7 nicotinic receptor agonist.

Nicotinic acetylcholine receptors (nAChRs) play a large role in central nervous system (CNS) activity and in different tissue throughout the body. They are known to be involved in functions, including, but not limited to, cognition, learning, mood, emotion, and neuroprotection. There are several types of nicotinic acetylcholine receptors, and each one appears to have a different role. Some nicotinic receptors regulate CNS function, including, but not limited to, attention, learning and memory; some regulate pain, inflammation, cancer, and diabetes by controlling tumor necrosis factor alpha (TNF-α). Nicotine affects all such receptors, and has a variety of activities. Unfortunately, not all of the activities are desirable. In fact, undesirable properties of nicotine include its addictive nature and the low ratio between efficacy and safety.

Schizophrenia is a complex multifactorial illness caused by genetic and non-genetic risk factors that produce a wide variety of symptoms. Historically, the disease has been characterized by positive and negative symptoms. The positive symptoms include delusions and hallucinations and the negative symptoms include apathy, withdrawal, lack of motivation and pleasure. More recently, deficits in affect, attention, cognition and information processing have been recognized as key pathologies in this complex disorder. No single biological element has emerged as a dominant pathogenic factor in this disease. Indeed, it is likely that schizophrenia is a syndrome that is produced by the combination of many low penetrance risk factors. Pharmacological studies established that dopamine receptor antagonists are efficacious in treating the overt psychotic features (positive symptoms) of schizophrenia such as hallucinations and delusions. Clozapine, an “atypical” antipsychotic drug, is novel because it is effective in treating not only the positive symptoms, but also negative, and to some extent the cognitive symptoms of this disease. Clozapine's utility as a drug is greatly limited because continued use leads to an increased risk of agranulocytosis and seizure. No other antipsychotic drug is effective in treating the cognitive symptoms of schizophrenia. This is significant because the restoration of cognitive functioning is the best predictor of a successful clinical and functional outcome of schizophrenic patients (Green, M. F., Am J. Psychiatry, 153:321-30, 1996).

By extension, it is clear that better drugs are needed to treat the cognitive disorders of schizophrenia in order to restore a better state of mental health to patients with this disorder. One aspect of the cognitive deficit of schizophrenia can be measured by using the auditory event-related potential (P50) test of sensory gating. In this test, electroencepholographic (EEG) recordings of neuronal activity of the hippocampus are used to measure the subject's response to a series of auditory “clicks” (Adler, L. E. et. al., Biol. Psychiatry, 46:8-18, 1999). Normal individuals respond to the first click with greater degree than to the second click. In general, schizophrenics and schizotypal patients respond to both clicks nearly the same (Cullum, C. M. et. al., Schizophr. Res., 10:131-41, 1993). These data reflect a schizophrenic's inability to “filter” or ignore unimportant information. The sensory transiently gating deficit appears to be one of the key pathological features of this disease (Cadenhead, K. S. et. al., Am. J. Psychiatry, 157:55-9, 2000). Multiple studies show that nicotine normalizes the sensory deficit of schizophrenia (Adler, L. E. et. al., Am. J. Psychiatry, 150:1856-61, 1993). Pharmacological studies indicate that nicotine's effect on sensory gating is via the α7 nAChR (Adler, L. E. et. al., Schizophr. Bull., 24:189-202, 1998). Indeed, the biochemical data indicate that schizophrenics have 50% fewer of α7 nAChR receptors in the hippocampus, thus giving a rationale to partial loss of α7 nAChR functionality (Freedman, R. et. al., Biol. Psychiatry, 38:22-33, 1995). Interestingly, genetic data indicate that a polymorphism in the promoter region of the α7 nAChR gene is strongly associated with the sensory gating deficit in schizophrenia (Freedman, R. et. al., Proc. Nat'l Acad. Sci. USA, 94(2):587-92, 1997; Myles-Worsley, M. et. al., Am. J. Med. Genet, 88(5):544-50, 1999). To date, no mutation in the coding region of the α7 nAChR has been identified. Thus, schizophrenics express the same α7 nAChR as non-schizophrenics. Selective α7 nAChR agonists may be found using a functional assay on FLIPR (see WO 00/73431). FLIPR is designed to read the fluorescent signal from each well of a 96 or 384 well plate as fast as twice a second for up to 30 minutes. This assay may be used to accurately measure the functional pharmacology of α7 nAChR. To conduct such an assay, one uses cell lines that express functional forms of the α7 nAChR using the α7/5-HT₃ channel as the drug target and cell lines that express functional 5HT₃R. In both cases, the ligand-gated ion channel was expressed in SH-EP1 cells. Both ion channels can produce robust signal in the FLIPR assay.

Bray, C, et al., “Mice Deficient in CHRNA7, a Subunit of the Nicotinic Acetylcholine Receptor, Produce Sperm with Impaired Motility”, Biol. Reprod. Jun. 8, 2005, report genetic evidence that sperm nicotinic acetylcholine receptors are important for maintenance of normal sperm motility.

Metz, Christine N., et al., 6 Nature Immunol. No 8, 756-757, 2005, and de Jonge, Wouter J., 6 Nature Immunol. No. 8., 844-851, 2005, report that acetylcholine released by stimulation of the vagus nerve binds to alpha 7 nAChRs expressed by macrophages to suppress proinflammatory cytokine production. The authors indicate that the anti-inflammatory pathway can be manipulated with cholinaergic agonists such as nicotine, providing possible therapeutic approaches for treating postoperative ileus or controlling host inflammatory responses during sepsis.

α7 nicotinic receptor agonists are also described in U.S. Pat. Nos. 6,809,094, and 6,881,734, both of which are incorporated by reference herein in their entirety.

Pharmaceutical compositions comprising an α7 nicotinic receptor agonist and an antipsychotic drug are described in US Published App. 2003/045540, which is incorporated by reference herein in its entirety.

The compositions of the present invention that contain an α7 nicotinic receptor agonist are useful for the treatment of cognitive deficits or impairments in schizophrenia and in Alzheimer's Disease.

### SUMMARY OF THE INVENTION

The present invention relates to compounds of the Formula I 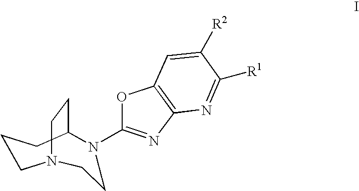
wherein

R¹ is selected from the group consisting of —CN, (C₁-C₈) alkyl, (C₃-C₈) cycloalkyl, 3-8 membered heterocycloalkyl, (C₆-C₁₀) aryl, -5-12 membered heteroaryl, OR³, —C(═O)NR³R⁴, —NR³C(═O)R⁴, —S(═O)₂R³, —S(═O)₂NR³R⁴, —NR³R⁴, wherein each said alkyl, cycloalkyl, heterocycloalkyl, aryl and heteroaryl is optionally substituted with one or more substituents, independently selected from F, Cl, Br, I, nitro, CN, CF₃, —NR⁹R¹⁰, —NR⁹C(═O)R¹⁰, —OR⁹, —C(═O)OR⁹, —C(═O)R⁹, —C(═O)NR⁹R¹⁰, —S(═O)₂NR⁹R¹⁰ and R⁹;

R² is selected from the group consisting of F, Cl, Br, I, nitro, —CN, CF₃, (C₁-C₈) alkyl, (C₃-C₈) cycloalkyl, 3-8 membered heterocycloalkyl, (C₆-C₁₀) aryl, and 5-12 membered heteroaryl, —NR⁶R⁷, —NR⁶C(═O)R⁷, —NR⁶S(═O)₂R⁷, —OR⁶, —OC(═O)R⁶, —C(═O)OR⁶, —C(═O)R⁶, —C(═O)NR⁶R⁷, —SR⁶, —S(═O)R⁶, —S(═O)₂R⁶, —S(═O)₂NR⁶R⁷; wherein each said alkyl, cycloalkyl, heterocycloalkyl, aryl and heteroaryl is optionally substituted with one or more substituents, independently selected from F, Cl, Br, I, nitro, CN, CF₃, —NR⁹R¹⁰, —NR⁹C(═O)R¹⁰, —OR⁹, —C(═O)OR⁹, —C(═O)R⁹, —C(═O)NR⁹R¹⁰, —SR⁹, —S(═O)R⁹, —S(═O)₂R⁹, —S(═O)₂NR⁹R¹⁰ and R⁹;

each of R³, R⁴, R⁶ and R⁷ is independently selected from H, (C₁-C₈)alkyl, (C₃-C₈)cycloalkyl, 3-8 membered heterocycloalkyl, (C₆-C₁₀) aryl, and 5-12 membered heteroaryl; wherein each of R³, R⁴, R⁶, and R⁷ is optionally substituted with one to six substituents, independently selected from F, Cl, Br, I, nitro, cyano, CF₃, —NR⁹R¹⁰, —NR⁹C(═O)R¹⁰, —NR⁹S(═O)₂R¹⁰, —OR⁹, —OC(═O)R⁹, —C(═O)OR⁹, —C(═O)R⁹, —C(═O)NR⁹R¹⁰, —SR⁹, —S(═O)R⁹, —S(═O)₂R⁹, —S(═O)₂NR⁹R¹ and R⁹;

or R³ and R⁴ taken together with the nitrogen of NR³R⁴ form a 3-8 membered heterocycloalkyl;

or R⁶ and R⁷ taken together with the nitrogen of NR⁶R⁷ form a 3-8 membered heterocycloalkyl;

each of R⁹ and R¹⁰ is independently selected from H, (C₁-C₈)alkyl, (C₃-C₈)cycloalkyl, 3-8 membered heterocycloalkyl, (C₆-C₁₀) aryl or 5-12 membered heteroaryl; wherein each of R⁹ and R¹⁰ is optionally substituted with one to six substituents independently selected from F, Cl, Br, I, nitro, cyano, CF₃, —NR¹²R¹³, —NR¹²C(═O)R¹³, —NR¹²S(═O)₂R¹³, —OR¹², C(═O)NR¹²R¹³, —SR¹², —S(═O)R¹², —S(═O)₂R², —S(═O)₂NR¹²R¹³ and R¹²;

or R⁹ and R¹⁰ taken together with the nitrogen of NR⁹R¹⁰ form a 3-8 membered heterocycloalkyl;

each of R¹² and R¹³ is independently selected from H, (C₁-C₈)alkyl, (C₃-C₈)cycloalkyl, 3-8 membered heterocycloalkyl, (C₆-C₁₀) aryl and (5-12 membered) heteroaryl;

or enantiomeric, diastereomeric, or tautomeric isomers thereof or pharmaceutically acceptable salts thereof.

More specific embodiments of this invention relate to compounds of the formula I wherein R¹ is (C₁-C₈) alkyl, (C₃-C₈) cycloalkyl, 3-8 membered heterocycloalkyl, (C₆-C₁₀) aryl, -5-12 membered heteroaryl, wherein each said alkyl, cycloalkyl, heterocycloalkyl, aryl and heteroaryl is optionally substituted with one or more substituents, independently selected from F, Cl, Br, I, nitro, CN, CF₃, —NR⁹R¹⁰, —OR⁹, and R⁹;

More specific embodiments of this invention relate to compounds of the formula I wherein R¹ is (C₁-C₈) alkyl wherein said alkyl is optionally substituted with one or more substituents independently selected from the group consisting of F, Cl, Br, I, nitro, CN, CF₃, —NR⁹R¹⁰, —OR⁹, and R⁹.

More specific embodiments of this invention relate to compounds of the formula I wherein R¹ is (C₆-C₁₀) aryl or 5-12 membered heteroaryl, wherein each of said aryl and heteroaryl is optionally substituted with one or more substituents independently selected from the group consisting of F, Cl, Br, I, nitro, CN, CF₃, —NR⁹R¹⁰, —OR⁹, and R⁹.

More specific embodiments of this invention relate to compounds of the formula I wherein R¹ is (C₁-C₈) alkyl. More specific embodiments of this invention relate to compounds of the formula I wherein R² is selected from the group consisting of F, Cl, Br, I, nitro, —CN, CF₃, (C₁-C₈)alkyl, (C₆-C₁₀) aryl, and 5-12 membered heteroaryl, —NR⁶R⁷, OR⁶ wherein each said alkyl, cycloalkyl, heterocycloalkyl, aryl and heteroaryl is optionally substituted with one or more substituents, independently selected from F, Cl, Br, I, nitro, CN, CF₃, —NR⁹R¹⁰, —NR⁹C(═O)R¹⁰, —OR⁹, —C(═O)OR⁹, —C(═O)R⁹, —C(═O)NR⁹R¹⁰, —SR⁹, —S(═O)R⁹, —S(═O)₂R⁹, —S(═O)₂NR⁹R¹⁰ and R⁹.

More specific embodiments of this invention relate to compounds of the formula I wherein R² is selected from the group consisting of —NR⁶R⁷, —NO₂, F, Cl, Br, I, —CN, (C₁-C₆) alkyl, (C₆-C₁₀) aryl, and O—(C₆-C₁₀) aryl.

More specific embodiments of the invention relate to compounds of the formula I wherein R¹ is (C₁-C₈) alkyl and R² is selected from the group consisting of F, Cl, Br, I, nitro, —CN, CF₃, (C₁-C₈)alkyl, (C₆-C₁₀) aryl, and 5-12 membered heteroaryl, —NR⁶R⁷, OR⁶ wherein each said alkyl, cycloalkyl, heterocycloalkyl, aryl and heteroaryl is optionally substituted with one or more substituents, independently selected from F, Cl, Br, I, nitro, CN, CF₃, —NR⁹R¹⁰, —NR⁹C(═O)R¹⁰, —OR⁹, —C(═O)OR⁹, —C(═O)R⁹, —C(═O)NR⁹R¹⁰, —SR⁹, —S(═O)R⁹, —S(═O)₂R⁹, —S(═O)₂NR⁹R¹⁰ and R⁹.

More specific embodiments of the invention relate to compounds of the formula I wherein R¹ is (C₁-C₁₀) alkyl and R² is selected from the group consisting of —NR⁶R⁷, —NO₂, F, Cl, Br, I, —CN, (C₁-C₆) alkyl, (C₆-C₁₀) aryl, and O—(C₆-C₁₀) aryl.

More specific embodiments of the invention relate to compounds of the formula I wherein R¹ is (C₁-C₈) alkyl and R² is selected from the group consisting of —NR⁶R⁷, —NO₂, F, Cl, Br, I, —CN, (C₁-C₆) alkyl, phenyl, and O-phenyl.

More specific embodiments of the invention relate to compounds of the formula I wherein R¹ is (C₆-C₁₀) aryl and R is selected from the group consisting of F, Cl, Br, I, nitro, —CN, CF₃, (C₁-C₈)alkyl, (C₆-C₁₀) aryl, and 5-12 membered heteroaryl, —NR⁶R⁷, OR⁶ wherein each said alkyl, cycloalkyl, heterocycloalkyl, aryl and heteroaryl is optionally substituted with one or more substituents, independently selected from F, Cl, Br, I, nitro, CN, CF₃, —NR⁹R¹⁰, —NR⁹C(═O)R¹⁰, —OR⁹, —C(═O)OR⁹, —C(═O)R⁹, —C(═O)NR⁹R¹⁰, —SR⁹, —S(═O)R⁹, —S(═O)₂R⁹, —S(═O)₂NR⁹R¹ and R⁹.

More specific embodiments of the invention relate compounds of the formula I wherein R¹ is (C₆-C₁₀) aryl and R is selected from the group consisting of —NR⁶R⁷, —NO₂, F, Cl, Br, I, —CN, (C₁-C₆) alkyl, and O—(C₆-C₁₀) aryl.

The term “alkyl”, as used herein, unless otherwise indicated, includes saturated monovalent hydrocarbon radicals having straight or branched moieties. Examples of alkyl groups include, but are not limited to, methyl, ethyl, propyl, isopropyl, and t-butyl.

The term “cycloalkyl”, as used herein, unless otherwise indicated, includes non-aromatic saturated cyclic alkyl moieties wherein alkyl is as defined above. Examples of cycloalkyl include, but are not limited to, cyclopropyl, cyclobutyl, cyclopentyl, cyclohexyl, and cycloheptyl.

The term “aryl”, as used herein, unless otherwise indicated, includes an organic radical derived from an aromatic hydrocarbon by removal of one hydrogen atom. Examples of aryl groups include, but are not limited to phenyl and naphthyl.

The terms “heterocyclic” and “heterocycloalkyl”, as used herein, refer to non-aromatic cyclic groups containing one or more heteroatoms, preferably from one to four heteroatoms, each selected from O, S and N. The heterocyclic groups of this invention can also include ring systems substituted with one or more oxo moieties. Examples of non-aromatic heterocyclic groups include, but are not limited to, aziridinyl, azetidinyl, pyrrolidinyl, piperidinyl, azepinyl, piperazinyl, 1,2,3,6-tetrahydropyridinyl, oxiranyl, oxetanyl, tetrahydrofuranyl, tetrahydropyranyl, tetrahydrothienyl, tetrahydrothiopyranyl, piperidino, morpholino, thiomorpholino, thioxanyl, pyrrolinyl, indolinyl, 2H-pyranyl, 4H-pyranyl, dioxanyl, 1,3-dioxolanyl, pyrazolinyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, quinuclidinyl and quinolizinyl.

The term “heteroaryl”, as used herein, refers to aromatic groups containing one or more heteroatoms (O, S, or N). A multicyclic group containing one or more heteroatoms wherein at least one ring of the group is aromatic is a “heteroaryl” group. The heteroaryl groups of this invention can also include ring systems substituted with one or more oxo moieties. Examples of heteroaryl groups include, but are not limited to, pyridinyl, pyridazinyl, imidazolyl, pyrimidinyl, pyrazolyl, triazolyl, pyrazinyl, quinolyl, isoquinolyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, purinyl, oxadiazolyl, thiazolyl, thiadiazolyl, furazanyl, benzofurazanyl, benzothiophenyl, benzotriazolyl, benzothiazolyl, benzoxazolyl, quinazolinyl, quinoxalinyl, naphthyridinyl, dihydroquinolyl, tetrahydroquinolyl, dihydroisoquinolyl, tetrahydroisoquinolyl, benzofuryl, furopyridinyl, pyrolopyrimidinyl, and azaindolyl.

The foregoing heteroaryl, heterocyclic and heterocycloalkyl groups may be C-attached or N-attached (where such is possible). For instance, a group derived from pyrrole may be pyrrol-1-yl (N-attached) or pyrrol-3-yl (C-attached).

Examples of specific compounds of this invention are the following compounds of the formula I and their pharmaceutically acceptable salts, hydrates, solvates and optical and other stereoisomers:
4-(6-bromo-5-methyloxazolo[4,5-b]pyridin-2-yl)-1,4-diaza-bicyclo[3.2.2]nonane;
4-(6-Bromo-5-ethyl-oxazolo[4,5-b]pyridin-2-yl)-1,4-diaza-bicyclo[3.2.2]nonane;
4-(5,6-dimethyloxazolo[4,5-b]pyridin-2-yl)-1,4-diaza-bicyclo[3.2.2]nonane;
4-(6-Methyl-5-ethyl-oxazolo[4,5-b]pyridin-2-yl)-1,4-diaza-bicyclo[3.2.2]nonane;
4-(5-Methyl-6-nitro-oxazolo[4,5-b]pyridin-2-yl)-1,4-diaza-bicyclo[3.2.2]nonane;
2-(1,4-Diaza-bicyclo[3.2.2]non-4-yl)-5-methyl-oxazolo[4,5-b]pyridin-6-ylamine;
4-(6-Fluoro-5-methyl-oxazolo[4,5-b]pyridin-2-yl)-1,4-diaza-bicyclo[3.2.2]nonane;
4-(6-Chloro-5-methyl-oxazolo[4,5-b]pyridin-2-yl)-1,4-diaza-bicyclo[3.2.2]nonane;
4-(6-Chloro-5-ethyl-oxazolo[4,5-b]pyridin-2-yl)-1,4-diaza-bicyclo[3.2.2]nonane;
4-(5-methyl-6-phenyloxazolo[4,5-b]pyridin-2-yl)-1,4-diaza-bicyclo[3.2.2]nonane;
4-(5-methyl-6-phenoxyoxazolo[4,5-b]pyridin-2-yl)-1,4-diaza-bicyclo[3.2.2]nonane and
2-(1,4-diaza-bicyclo[3.2.2]nonan-4-yl)-5-methyloxazolo[4,5-b]pyridine-6-carbonitrile.

Unless otherwise indicated, the term “one or more substituents”, as used herein, refers to from one to the maximum number of substituents possible based on the number of available bonding sites.

The term “treatment”, as used herein, refers to reversing, alleviating, inhibiting the progress of, or preventing the disorder or condition to which such term applies, or one or more symptoms of such condition or disorder. The term “treatment”, as used herein, refers to the act of treating, as “treating” is defined immediately above.

Compounds of formula I may contain chiral centers and therefore may exist in different enantiomeric and diastereomeric forms. Individual isomers can be obtained by known methods, such as resolution, stereoselective reaction, or chromatographic separation in the preparation of the final product or its intermediate. This invention relates to all optical isomers and all stereoisomers of compounds of the formula I, both as racemic mixtures and as individual enantiomers and diastereoismers of such compounds, and mixtures thereof, and to all pharmaceutical compositions and methods of treatment defined above that contain or employ them, respectively.

In so far as the compounds of formula I of this invention are basic compounds, they are all capable of forming a wide variety of different salts with various inorganic and organic acids. Although such salts must be pharmaceutically acceptable for administration to animals, it is often desirable in practice to initially isolate the base compound from the reaction mixture as a pharmaceutically unacceptable salt and then simply convert to the free base compound by treatment with an alkaline reagent and thereafter convert the free base to a pharmaceutically acceptable acid addition salt. The acid addition salts of the base compounds of this invention are readily prepared by treating the base compound with a substantially equivalent amount of the chosen mineral or organic acid in an aqueous solvent or in a suitable organic solvent, such as methanol or ethanol. Upon careful evaporation of the solvent, the desired solid salt is readily obtained. The acids which are used to prepare the pharmaceutically acceptable acid addition salts of the aforementioned base compounds of this invention are those which form non-toxic acid addition salts, i.e., salts containing pharmaceutically acceptable anions, such as the chloride, bromide, iodide, nitrate, sulfate or bisulfate, phosphate or acid phosphate, acetate, lactate, citrate or acid citrate, tartrate or bitartrate, succinate, maleate, fumarate, gluconate, saccharate, benzoate, methanesulfonate, ethanesulfonate, benzenesulfonate, p-toluenesulfonate and pamoate (i.e., 1,1′-methylene-bis-(2-hydroxy-3-naphthoate))salts.

The present invention also includes isotopically labelled compounds, which are identical to those recited in formula I, but for the fact that one or more atoms are replaced by an atom having an atomic mass or mass number different from the atomic mass or mass number usually found in nature. Examples of isotopes that can be incorporated into compounds of the present invention include isotopes of hydrogen, carbon, nitrogen, oxygen, phosphorous, sulfur, fluorine and chlorine, such as ²H, ³H, ¹³C, ¹¹C, ¹⁴C, ¹⁵N, ¹⁸O, ¹⁷O, ³¹P, ³²P, ³⁵S, ¹⁸F, ³⁶Cl, and ¹²³I, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of formula I of this invention and prodrugs thereof can generally be prepared by carrying out the procedures disclosed in the Schemes and/or in the Examples and Preparations below, by substituting a readily available isotopically labelled reagent for a non-isotopically labelled reagent.

The present invention also relates to a pharmaceutical composition comprising a compound of the formula I, or a pharmaceutically acceptable salt thereof, and a pharmaceutically acceptable carrier.

The present invention also relates to a pharmaceutical composition for the treatment of schizophrenia in a mammal, including a human, comprising an amount of a compound of the formula I, or a pharmaceutically acceptable salt thereof, that is effective in treating schizophrenia and a pharmaceutically acceptable carrier.

The present invention also relates to a method for treating schizophrenia in a mammal, including a human, comprising administering to said mammal an amount of a compound of the formula I, or a pharmaceutically acceptable salt thereof, that is effective in treating schizophrenia.

The present invention also relates to a pharmaceutical composition for the treatment of schizophrenia in a mammal, including a human, comprising an α7 nicotinic receptor agonizing amount of a compound of formula I and a pharmaceutically acceptable carrier.

The present invention also relates to a method for treating schizophrenia in a mammal, including a human, comprising administering to said mammal an α7 nicotinic receptor agonizing amount of a compound of the formula I, or a pharmaceutically acceptable salt thereof.

This invention provides a method of treating a disorder or condition comprising as a symptom a deficiency in attention and/or cognition in a mammal, including a human, which method comprises administering to said mammal an amount of a compound of Formula I or a pharmaceutically acceptable salt thereof effective in treating said disorder or condition.

A deficiency in attention and/or cognition in connection with a disorder comprising as a symptom a deficiency in attention and/or cognition refers to a subnormal functioning in one or more cognitive aspects such as memory, intellect, or learning and logic ability, in a particular individual relative to other individuals within the same general age population. A deficiency in attention and/or cognition also refers to a reduction in any particular individual's functioning in one or more cognitive aspects.

This invention further provides a method of treating a neurodegenerative disorder or condition in a mammal, including a human, which method comprises administering to said mammal an amount of a compound of Formula I or a pharmaceutically acceptable salt thereof effective in treating said disorder or condition.

As used herein, and unless otherwise indicated, a neurodegenerative disorder or condition refers to a disorder or condition that is caused by the dysfunction and/or death of neurons in the central nervous system. The treatment of these disorders and conditions can be facilitated by administration of an agent which prevents the dysfunction or death of neurons at risk in these disorders or conditions and/or enhances the function of damaged or healthy neurons in such a way as to compensate for the loss of function caused by the dysfunction or death of at-risk neurons.

A neurodegenerative disorder that can be treated according to the present invention includes, but is not limited to, Alzheimer's Disease.

The compounds of Formula I are useful to treat, or are useful to make a medicament to treat, a condition in a mammal that may be treated by administration of an α7 nicotinic acetylcholine receptor agonist. The compounds of Formula I are useful to treat, or are useful to make a medicament to treat, a mammal where the mammal receives symptomatic relief from activation of an α7 nicotinic acetylcholine receptor agonist.

For example, the present invention also relates to a pharmaceutical composition for treating a disorder or condition selected from cognitive and attention deficit symptoms of Alzheimer's, neurodegeneration associated with diseases such as Alzheimer's disease, pre-senile dementia (mild cognitive impairment), senile dementia, schizophrenia or psychosis including the cognitive deficits associated therewith, attention deficit disorder, attention deficit hyperactivity disorder (ADHD), mood and affective disorders, amyotrophic lateral sclerosis, borderline personality disorder, traumatic brain injury, behavioral and cognitive problems associated with brain tumors, AIDS dementia complex, dementia associated with Down's syndrome, dementia associated with Lewy Bodies, Huntington's disease, depression, general anxiety disorder, age-related macular degeneration, Parkinson's disease, tardive dyskinesia, Pick's disease, post traumatic stress disorder, dysregulation of food intake including bulemia and anorexia nervosa, withdrawal symptoms associated with smoking cessation and dependent drug cessation, Tourette's syndrome, glaucoma, neurodegeneration associated with glaucoma, symptoms associated with pain, pain and inflammation, TNF-α related conditions, rheumatoid arthritis, rheumatoid spondylitis, muscle degeneration, osteoporosis, osteoarthritis, psoriasis, contact dermatitis, bone resorption diseases, atherosclerosis, Paget's disease, uveititis, gouty arthritis, inflammatory bowel disease, adult respiratory distress syndrome (ARDS), Crohn's disease, rhinitis, ulcerative colitis, anaphylaxis, asthma, Reiters syndrome, tissue rejection of a graft, ischemia reperfusion injury, stroke, multiple sclerosis, cerebral malaria, sepsis, septic shock, toxic shock syndrome, fever and myalgias due to infection, HIV-1, HIV-2, and HIV-3, cytomegalovirus (CMV), influenza, adenovirus, a herpes virus (including HSV-1, HSV-2), a herpes zoster, cancer (multiple myeloma, acute and chronic myelogenous leukemia, or cancer-associated cachexia), diabetes (pancreatic beta cell destruction, or type I and type II diabetes), wound healing (healing burns, and wounds in general including from surgery), bone fracture healing, ischemic heart disease, tinnitus, or stable angina pectoris in a mammal, comprising an amount of a compound of the formula I, or a pharmaceutically acceptable salt thereof, that is effective in treating such disorder or condition and a pharmaceutically acceptable carrier. A condition that is preferred for treatment is attention deficit disorder, attention deficit hyperactivity disorder, mood and affective disorders, amyotrophic lateral sclerosis, borderline personality disorder, traumatic brain injury, behavioral and cognitive problems associated with brain tumors, AIDS dementia complex, dementia associated with Down's syndrome, dementia associated with Lewy Bodies, Huntington's disease, depression, general anxiety disorder, age-related macular degeneration, Parkinson's disease, tardive dyskinesia, Pick's disease, post traumatic stress disorder, dysregulation of food intake including bulemia and anorexia nervosa, withdrawal symptoms associated with smoking cessation and dependent drug cessation, Gilles de la Tourette's Syndrome, glaucoma, neurodegeneration associated with glaucoma, or symptoms associated with pain.

The present invention also relates to a pharmaceutical composition for treating male infertility.

The present invention also relates to a pharmaceutical composition for treating inflammation, for example, postoperative ileus.

The present invention also relates to a method for treating a disorder or condition listed, comprising administering to a mammal in need of such treatment an amount of a compound of the formula I, or a pharmaceutically acceptable salt thereof, that is effective in treating such disorder or condition.

The present invention also relates to a pharmaceutical composition, which may be a composition for treating a disorder or condition listed in the previous paragraphs, comprising an α7 nicotinic receptor agonizing amount of a compound of the formula I, or a pharmaceutically acceptable salt thereof, and a pharmaceutically acceptable carrier.

The present invention also relates to a method for treating a disorder or condition listed in the previous paragraphs, comprising administering to a mammal in need of such treatment an α7 nicotinic receptor agonizing amount of a compound of the formula I, or a pharmaceutically acceptable salt thereof.

The present invention also relates to a method for treating a disease or condition in a mammal in need thereof, wherein the mammal receives symptomatic relief from activation of an α7 nicotinic acetylcholine receptor, comprising administering to a mammal in need of such treatment a compound of the formula I, or a pharmaceutically acceptable salt thereof. The disease or condition may be, for example, cognitive and attention deficit symptoms of Alzheimer's, neurodegeneration associated with diseases such as Alzheimer's disease, pre-senile dementia (mild cognitive impairment), or senile dementia. The disease or condition may also be, for example, schizophrenia or psychosis and related cognitive deficits associated therewith. The disease or condition may also be, for example, attention deficit disorder, attention deficit hyperactivity disorder, mood and affective disorders, amyotrophic lateral sclerosis, borderline personality disorder, traumatic brain injury, behavioral and cognitive problems associated with brain tumors, AIDS dementia complex, dementia associated with Down's syndrome, dementia associated with Lewy Bodies, Huntington's disease, depression, general anxiety disorder, age-related macular degeneration, Parkinson's disease, tardive dyskinesia, Pick's disease, post traumatic stress disorder, dysregulation of food intake including bulemia and anorexia nervosa, withdrawal symptoms associated with smoking cessation and dependent drug cessation, Gilles de la Tourette's Syndrome, glaucoma, neurodegeneration associated with glaucoma, or symptoms associated with pain.

The present invention also relates to a method for treating male infertility in a mammal in need thereof comprising administering to the mammal a compound of Formula I or a pharmaceutically acceptable salt thereof.

The present invention also relates to a method for treating inflammation such as postoperative ileus, in a mammal in need thereof comprising administering to the mammal a compound of Formula I or a pharmaceutically acceptable salt thereof.

The present invention also relates to a pharmaceutical composition comprising a compound of the formula I, or a pharmaceutically acceptable salt thereof, and an antipsychotic drug or pharmaceutically acceptable salt thereof.

The present invention also relates to a method of treating a mammal suffering from schizophrenia or psychosis, comprising administering a compound of formula I, or a pharmaceutically acceptable salt thereof, in an amount that is effective in treating schizophrenia, and an antipsychotic drug or pharmaceutically acceptable salt thereof. The compound of formula I and the antipsychotic drug may be administered together or separately. The compound of formula I and the antipsychotic drug may be administered simultaneously or at separate intervals. When administered simultaneously the compound of formula I and the antipsychotic drug may be incorporated into a single pharmaceutical composition. Alternatively, two separate compositions, i.e., one containing a compound of formula I and the other containing an antipsychotic drug, may be administered simultaneously.

The antipsychotic drug may be, for example, Chlorpromazine, Fluphenazine, Haloperidol, Loxapine, Mesoridazine, Molindone, Perphenazine, Pimozide, Thioridazine, Thiothixene, or Trifluoperazine. These drugs all have an affinity for the dopamine 2 receptor. The antipsychotic drug may also be, for example, Asenapine, Ziprasidone, Olanzapine, Clozapine, Risperidone, Sertindole, Quetiapine, Aripiprazole or Amisulpride.

Certain combinations of this invention include at least two active components: an atypical antipsychotic, a prodrug thereof, a pharmaceutically acceptable salt thereof, or a pharmaceutically acceptable salt of said prodrug, and a compound of Formula I or a pharmaceutically acceptable salt thereof. The combinations of this invention also include a pharmaceutically acceptable vehicle, carrier or diluent.

The combinations may result in synergistic action allowing a lower dose of the atypical anti psychotic to be administered while achieving at least the same psychotropic effect as achieved with a standard dose of the atypical antipsychotic. The dosage of the atypical antipsychotic may be reduced by about 25-90%, for example, about 40-80% and typically about 50-70%. The reduction in amount of antipsychotic required will be dependent on the amount of the compound of Formula I given.

The selection of the dosage of each therapeutic agent is that which can provide relief to the patient as measured by a reduction or amelioration of symptoms associated with the disorder or condition of the patient. As is well known, the dosage of each component depends on several factors such as the potency of the selected specific compound, the mode of administration, the age and weight of the patient, the severity of the condition to be treated, and the like. Determining a dose is within the skill of the ordinary artisan. To the extent necessary for completeness, the synthesis of the components of the compositions and dosages are as described in the listed patents above or the Physicians' Desk Reference, 57th ed., Thompson, 2003 which are expressly incorporated herein by reference. Desirably, when ziprasidone is selected as the active agent, the daily dose contains from about 5 mg to about 460 mg. More preferably, each dose of the first component contains about 20 mg to about 320 mg of the ziprasidone, and even more preferably, each dose contains from about 20 mg to about 160 mg of ziprasidone. Pediatric dosages may be less such as for example in the range of about 0.5 mg to about 40 mg daily. This dosage form permits the full daily dosage to be administered in one or two oral doses, for example.

General outlines of the dosages for the atypical antipsychotics, and some preferred dosages, are provided herein. This list is not intended to be complete but is merely a guideline for any of the desired combinations of the present invention.

Olanzapine: from about 0.25 to about 100 mg, once/day; preferably, from about 1 to about 30 mg, once/day; and most preferably about 1 to about 25 mg once/day;

Clozapine: from about 12.5 to about 900 mg daily; preferably, from about 150 to about 450 mg daily;

Risperidone: from about 0.25 to about 16 mg daily; preferably, from about 2-8 mg daily;

Sertindole: from about 0.0001 to about 1.0 mg/kg daily;

Quetiapine: from about 1.0 to about 40 mg/kg given once daily or in divided doses;

Asenapine: from about 0.005 to about 60 mg total per day, given as a single dose or in divided doses;

Paliperidone: from about 0.01 mg/kg to about 4 mg/kg body weight, more preferably from about 0.04 to about 2 mg/kg body weight;

Bifeprunox.

The presently preferred atypical antipsychotic used according to the invention is ziprasidone. Ziprasidone (5-[2-[4-(1,2-benzisothiazol-3-yl)piperazin-1-yl]ethyl]-6-chloroindolin-2-one) is a benzisothiazolyl piperazine atypical antipsychotic with in vitro activity as a 5-HT_{1A} receptor agonist and an inhibitor of serotonin and norepinephrine reuptake (U.S. Pat. No. 4,831,031). The postsynaptic 5-HT_{1A} receptor has been implicated in both depressive and anxiety disorders (N M Barnes, T Sharp, 38 Neuropharmacology 1083-152, 1999). Oral bioavailability of ziprasidone taken with food is approximately 60%, half-life is approximately 6-7 hours, and protein binding is extensive.

Ziprasidone is efficacious for the treatment of patients with schizophrenia and schizomood disorders, refractory schizophrenia, cognitive impairment in schizophrenia, affective and anxiety symptoms associated with schizoaffective disorder and bipolar disorder. The drug is considered a safe and efficacious atypical antipsychotic (Charles Caley & Chandra Cooper, 36 Ann. Pharmacother., 839-51; (2002).

The present invention is useful in treating mental disorders and conditions, the treatment of which is facilitated by the administration of ziprasidone. Thus, the present invention has application where ziprasidone use is indicated as, e.g., in U.S. Pat. Nos. 6,245,766; 6,245,765; 6,387,904; 5,312,925; 4,831,031; and European EP 0901789 published Mar. 17, 1999, all of which are incorporated herein by reference.

Other atypical antipsychotics which can be used include, but are not limited to: Olanzapine, 2-methyl-4-(4-methyl-1-piperazinyl)-10H-thieno[2,3-b][1,5]benzodiazepine. Olanizapine is a known compound and is described in U.S. Pat. No. 5,229,382 as being useful for the treatment of schizophrenia, schizophreniform disorder, acute mania, mild anxiety states, and psychosis. U.S. Pat. No. 5,229,382 is herein incorporated herein by reference in its entirety;

Clozapine, 8-chloro-11-(4-methyl-1-piperazinyl)-5H-dibenzo[b,e][1,4]diazepine. Clozapine is described in U.S. Pat. No. 3,539,573, which is herein incorporated by reference in its entirety. Clinical efficacy in the treatment of schizophrenia is described (Hanes, et al., Psychopharmacol. Bull., 24, 62 (1988));

Risperidone, 3-[2-[4-(6-fluoro-1,2-benzisoxazol-3-yl)piperidino]ethyl]-2-methyl-6,7,8,9-tetrahydro-4H-pyrido-[1,2-a]pyrimidin-4-one. Risperidone and its use in the treatment of psychotic diseases are described in U.S. Pat. No. 4,804,663, which is herein incorporated by reference in its entirety;

Sertindole, 1-[2-[4-[5-chloro-1-(4-fluorophenyl)-1H-indol-3-yl]-1-piperidinyl]ethyl]imidazolidin-2-one. Sertindole is described in U.S. Pat. No. 4,710,500. Its use in the treatment of schizophrenia is described in U.S. Pat. Nos. 5,112,838 and 5,238,945. U.S. Pat. Nos. 4,710,500; 5,112,838; and 5,238,945 are herein incorporated by reference in their entireties;

Quetiapine, 5-[2-(4-dibenzo[b,f][1,4]thiazepin-11-yl-1-piperazinyl)ethoxy]ethanol. Quetiapine and its activity in assays which demonstrate utility in the treatment of schizophrenia are described in U.S. Pat. No. 4,879,288, which is herein incorporated by reference in its entirety. Quetiapine is typically administered as its (E)-2-butenedioate (2:1) salt.

Aripiprazole, 7-{4-[4-(2,3-dichlorophenyl)-1-piperazinyl]-butoxy}-3,4-dihydro carbostyril or 7-{4-[4-(2,3-dichlorophenyl)-1-piperazinyl]-butoxy}-3,4-dihydro-2(1H)-quinolinone. Aripiprazole is an atypical antipsychotic agent used for the treatment of schizophrenia and described in U.S. Pat. No. 4,734,416 and U.S. Pat. No. 5,006,528, which are herein incorporated by reference in their entireties.

Amisulpride, which is described in U.S. Pat. No. 4,401,822. U.S. Pat. No. 4,401,822 is incorporated herein in its entirety.

Asenapine, trans-5-chloro-2-methyl-2,3,3a,12b-tetrahydro-1H-dibenz[2,3:6,7]oxepino[4,5-c]pyrrole. Preparation and use of asenapine is described in U.S. Pat. Nos. 4,145,434 and 5,763,476, the entire contents of which are incorporated herein by reference.

Paliperidone, 3-[2-[4-(6-fluoro-1,2-benzisoxazol-3-yl)-1-piperidinyl]ethyl]-6,7,8,9-tetrahydro-9-hydroxy-2-methyl-4H-pyrido[1,2-a]pyrimidin-4-one. Preparation and use of paliparidone is described, for example, in U.S. Pat. Nos. 6,320,048; 5,158,952; and 5,254,556, the entire contents of which are incorporated herein by reference.

Bifeprunox, 2-[4-[4-(5-fluoro-1H-indol-3-yl)-3,6-dihydro-1 (2H)-pyridinyl]butyl]-1H-isoindole-1,3(2H)-dione. Preparation and use of bifeprunox is described in U.S. Pat. No. 6,225,312, which is incorporated in its entirety herein.

A preferred combination is ziprasidone with a compound of Formula I or a pharmaceutically acceptable salt of the present invention.

### DETAILED DESCRIPTION OF THE INVENTION

The compounds of the Formula I may be prepared by the methods described below, together with synthetic methods known in the art of organic chemistry, or modifications and derivatizations that are familiar to those of ordinary skill in the art. In the schemes and discussion that follow, R¹, R², R³, R⁴, R⁶ and R⁷ unless otherwise indicated, are defined as above in the definition of compounds of Formula I. Preferred methods include, but are not limited to, those described below.

The reactions described below are performed in solvents that are appropriate to the reagents and materials employed and that are suitable for use in the reactions described: In the description of the synthetic methods described below, it is also to be understood that all reaction conditions, whether actual or proposed, including choice of solvent, reaction temperature, reaction duration time, reaction pressure, and other reaction conditions (such as anhydrous conditions, under argon, under nitrogen, etc.), and work up procedures, are those conditions that are standard for that reaction, as would be readily recognized by one of skill in the art. Alternate methods that are known in the literature may also be used.

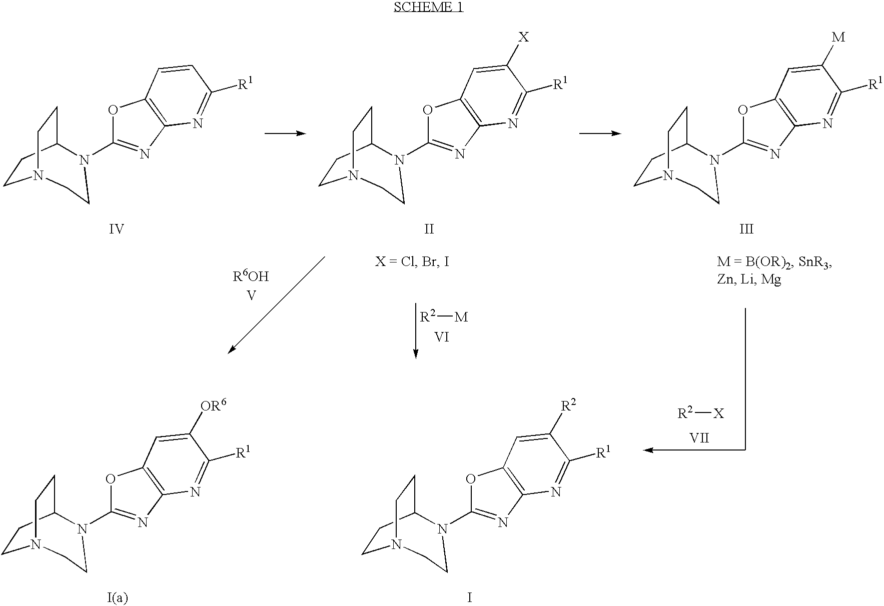

Referring to Scheme 1, reacting a compound of the formula IV with a halogenating reagent produces a compound of formula II where X is the corresponding halogen. The halogenating agent may be, but is not limited to, Cl₂, Br₂, I₂, N-bromosuccinimide, N-chlorosuccinimide, or N-iodosuccinimide. The reaction may be performed in an inert reaction solvent such as water, acetic acid, methanol, ethanol, tetrahydrofuran (THF), carbon tetrachloride, chloroform, acetonitrile or mixtures thereof in the presence or absence of a base such as potassium acetate, sodium acetate, cesium acetate, sodium carbonate, lithium carbonate, potassium carbonate, cesium carbonate, cesium fluoride n-butyllithium, lithium diisopropyl amide at −78° C. to 100° C. In an exemplary embodiment, reaction with Br₂ in water and acetic acid with sodium acetate at 25° C. to 100° C. produces a compound of formula II where X is Br.

Referring to Scheme 1, a compound of the formula I can be prepared from a compound of formula II wherein X is chloro, bromo, or iodo by first reacting it with bis(pinacolato)diboron and a palladium catalyst such as palladium (0) tetrakis(triphenylphosphine), palladium (II) acetate, allyl palladium chloride dimer, tris(dibenzylideneacetone)dipalladium (0), tris(dibenzylidene-acetone)dipalladium (0) chloroform adduct, palladium (II) chloride or dichloro[1,1′-bis(diphenylphosphino)ferrocene]palladium (II) dichloromethane adduct, preferably dichloro[1,1′-bis(diphenylphosphino)-ferrocene]palladium (II) dichloromethane adduct, in the presence or absence of a phosphine ligand such as 1,1′-bis(diphenylphosphino)ferrocene, triphenylphosphine, tri-o-tolylphosphine, tri-tert-butylphosphine, 1,2-bis(diphenylphosphino)ethane, 1,3-bis(diphenylphosphino)-propane, BINAP, 2-biphenyl dicyclohexylphosphine, 2-biphenyl-di-tert-butylphosphine, 2-(N,N-dimethylamino)-2′-di-tert-butylphosphino-biphenyl or 2-(N,N-dimethylamino)-2′-dicyclohexylphosphinobiphenyl, preferably 1,1′-bis(diphenylphosphino)ferrocene, and in the presence or absence of a base such as potassium acetate, sodium acetate, cesium acetate, sodium carbonate, lithium carbonate, potassium carbonate, cesium carbonate or cesium fluoride, preferably potassium acetate, to yield a compound of the formula III wherein the X group has been replaced with M, wherein M=borane pinacol ester. Generally, this reaction is carried out in a reaction inert solvent such as 1,4-dioxane, acetonitrile, methyl sulfoxide, tetrahydrofuran, ethanol, methanol, 2-propanol, toluene, preferably methyl sulfoxide, at a temperature from about from 0° C. to about 200° C., preferably from about 80° C. to about 120° C.

Other methods of converting a compound of the formula II with the X group mentioned above into a compound of the formula III wherein the X group is replaced with M, wherein M is boronic acid, boronic acid ester or trialkylstannane, are known in the art. For instance, treatment of a compound of the formula II, wherein X is Br or I, with an alkyl lithium reagent such as, but not limited to n-butyl lithium, sec butyl lithium or tert-butyl lithium, in a solvent such as diethyl ether, tetrahydrofuran, 1,2-dimethoxyethane, hexane, toluene, dioxane or a similar reaction inert solvent, at a temperature from −100° C. to 25° C. affords the corresponding compound of the formula III wherein X is Li. Treatment of a solution of this material with a suitable boronic ester such as trimethylborate, triethylborate or triisopropylborate, followed by a standard aqueous work-up with acid will afford the corresponding compound of the formula III wherein M is boronic acid.

Alternatively, treating a mixture of a compound of the formula II wherein X is Br or I and a boronic ester with an alkyl lithium reagent, as described above, followed by a standard aqueous work-up with acid will afford the corresponding compound of formula III wherein M is boronic acid. Alternatively, treating a compound of the formula II wherein X is Br or I with an alkyl lithium reagent such as, but not limited to n-butyl lithium, sec butyl lithium or tert-butyl lithium, in a solvent such as diethyl ether, tetrahydrofuran, dimethoxyethane, hexane, toluene, dioxane or a similar reaction inert solvent, at a temperature from a −100° C. to 25° C. will afford the corresponding compound of the formula III wherein M is Li. Treatment of a solution of this material with a suitable trialkylstannyl halide such as, but not limited to trimethylstannyl chloride or bromide or tributylstannyl chloride or bromide, followed by a standard aqueous work-up will afford the corresponding compound of the formula III wherein M is trimethyl or tributylstannane.

Referring to Scheme 1, reaction of a compound of the formula III wherein M is a boronic acid, boronic ester, or trialkylstannane group, with an aryl or heteroaryl chloride, aryl or heteroaryl bromide, aryl or heteroaryl iodide, or aryl or heteroaryl triflate of the formula VII, preferably an aryl or heteroaryl bromide, with a palladium catalyst such as palladium (0) tetrakis(triphenylphosphine), palladium (II) acetate, allyl palladium chloride dimer, tris(dibenzylideneacetone)dipalladium (0), tris(dibenzylideneacetone)dipalladium (0) chloroform adduct, palladium (II) chloride or dichloro[1,1′-bis(diphenylphosphino)ferrocene]palladium (II) dichloromethane adduct, preferably dichloro[1,1′-bis(diphenylphosphino)-ferrocene]palladium (II) dichloromethane adduct, in the presence or absence of a phosphine ligand such as 1,1′-bis(diphenylphosphino)ferrocene, triphenylphosphine, tri-o-tolylphosphine, tri-tert-butylphosphine, 1,2-bis(diphenylphosphino)ethane, 1,3-bis(diphenylphosphino)-propane, BINAP, 2-biphenyl dicyclohexylphosphine, 2-biphenyl-di-tert-butylphosphine, 2-(N,N-dimethylamino)-2′-di-tert-butylphosphino-biphenyl or 2-(N,N-dimethylamino)-2′-dicyclohexylphosphinobiphenyl, preferably 1,1′-bis(diphenylphosphino)ferrocene, and in the presence or absence of a base such as potassium phosphate, potassium acetate, sodium acetate, cesium acetate, sodium carbonate, lithium carbonate, potassium carbonate, cesium fluoride or cesium carbonate, preferably potassium phosphate, affords a compound of formula I. This reaction is typically carried out in a reaction inert solvent such as 1,4-dioxane, acetonitrile, methyl sulfoxide, tetrahydrofuran, ethanol, methanol, 2-propanol, or toluene, preferably 1,4-dioxane, in the presence or absence of from about 1%-about 10% water, preferably about 5% water, with or without microwave assisted heating at a temperature from about 0° C. to about 200° C., preferably from about 60° C. to about 100° C.

Referring to Scheme 1, alternatively, a compound of the formula II can be reacted with a compound of the formula VI to yield a compound of formula I, wherein M is a boronic acid, boronic acid ester, borane pinacol ester, zinc or trialkylstannane group, in the presence of a palladium catalyst such as palladium (0) tetrakis(triphenylphosphine), palladium (II) acetate, allyl palladium chloride dimer, tris(dibenzylideneacetone)dipalladium (0), tris(dibenzylideneacetone)dipalladium (0) chloroform adduct, palladium (II) chloride or dichloro[1,1′-bis(diphenylphosphino)ferrocene]palladium (II) dichloromethane adduct, preferably palladium (II) acetate, in the presence or absence of a phosphine ligand such as 1,1′-bis(diphenylphosphino)ferrocene, triphenylphosphine, tri-o-tolylphosphine, tri-tert-butylphosphine, 1,2-bis(diphenylphosphino)ethane, 1,3-bis(diphenylphosphino)-propane, BINAP, 2-biphenyl dicyclohexylphosphine, 2-biphenyl-di-tert-butylphosphine, 2-(N,N-dimethylamino)-2′-di-tert-butylphosphino-biphenyl or 2-(N,N-dimethylamino)-2′-dicyclohexylphosphinobiphenyl, preferably 2-(N,N-dimethylamino)-2′-dicyclohexylphosphinobiphenyl, and in the presence or absence of a base such as potassium phosphate, potassium acetate, sodium acetate, cesium acetate, sodium carbonate, lithium carbonate, potassium carbonate, cesium fluoride or cesium carbonate, preferably cesium fluoride, affords a compound of formula I. This reaction is typically carried out in a reaction inert solvent such as 1,4-dioxane, 1,2-dimethoxyethane, acetonitrile, methyl sulfoxide, tetrahydrofuran, ethanol, methanol, 2-propanol, or toluene, preferably 1,2-dimethoxyethane, in the presence or absence of from about 1% to about 10% triethylamine, preferably about 1% triethylamine, at a temperature from about 0° C. to about 200° C. with or without microwave assisted heating.

Referring to Scheme 1, alternatively, a compound of the formula II can be reacted with a compound of the formula V to yield a compound of formula I(a). The reaction can be carried out in the presence of a copper salt such as, but not limited to, copper(I) chloride (CuCl), copper(II) triflate and copper(I) iodide (CuI), in the presence or absence of a ligand such as, but not limited to, 2,2,6,6-tetramethylheptane-3,5-dione (TMHD), 1,10-phenanthroline, 8-hydroxyquinoline, 2-aminopyridine and pentane-2,4-dione (acac), and in the presence or absence of a base such as cesium carbonate, potassium phosphate, potassium acetate, sodium acetate, cesium acetate, sodium carbonate, lithium carbonate, potassium carbonate, preferably cesium carbonate, using the reacting alcohol as solvent or in an inert solvent such as, but not limited to, benzene, toluene, xylene, N,N-dimethylformamide (DMF), dimethylsulfoxide (DMSO) and N-methylpyrrolidinone (NMP) at a temperature from about 0° C. to about 200° C.

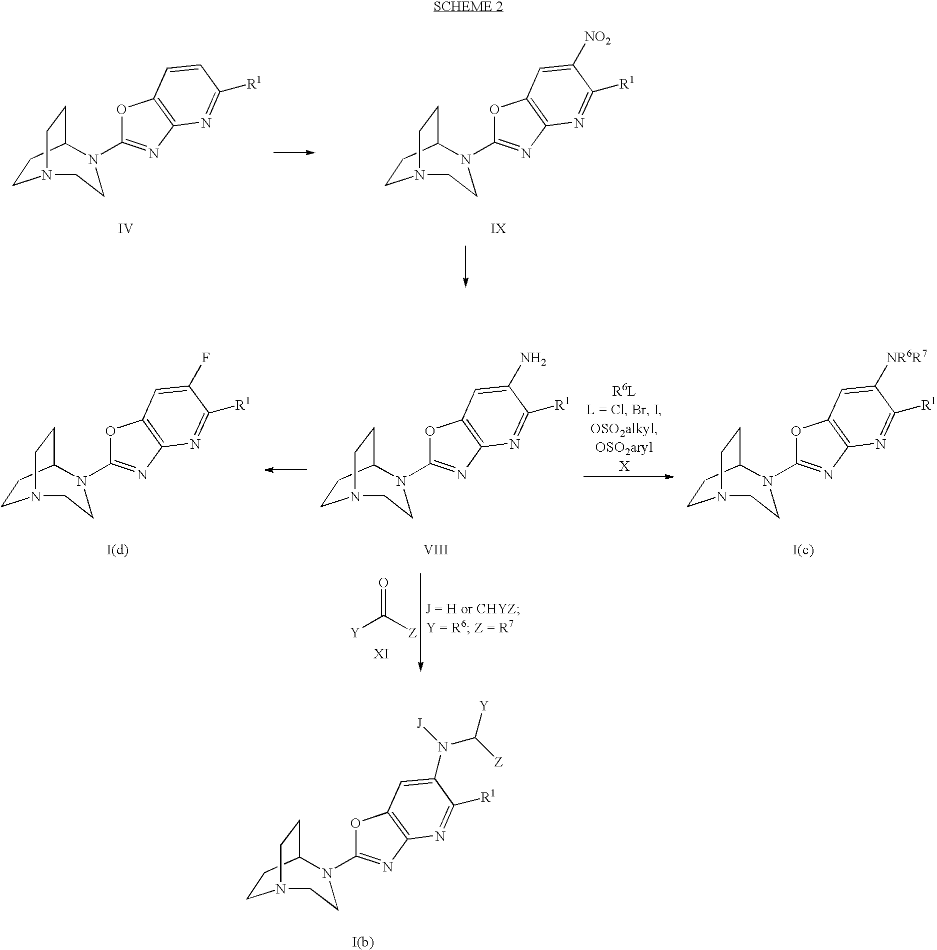

Compounds of the formula I can be prepared as illustrated in Scheme 2. Referring to Scheme 2, reacting a compound of formula IX, which can be obtained, for example, by nitration of a compound of formula IV, under reducing conditions such as but not limited to zinc, tin or iron and acid, catalytic hydrogenation, transfer hydrogenolysis or sodium hydrosulfite in an inert reaction solvent such as water, methanol, ethanol, isopropanol, with the preferred conditions being catalytic hydrogenation using palladium on carbon as a catalyst in ethanol at ambient temperature and 50 psi of hydrogen, affords a compound of formula VIII wherein the nitro group is converted to a primary amine. The compound of formula VIII can then be treated with a compound of formula XI wherein Y and Z are defined as R⁶ and R⁷ above, except that neither Y nor Z may be hydrogen, and a reducing agent such as but not limited to sodium triacetoxyborohydride, sodium borohydride, sodium cyanoborohydride, lithium aluminum hydride, catalytic hydrogenation or transfer hydrogenolysis in the presence or absence of an acid such as but not limited to acetic acid, hydrochloric acid, trifluoroacetic acid, sulfuric acid, phosphoric acid or nitric acid in an inert reaction solvent such as chloroform, dichloromethane, 1,2-dichloroethane, acetonitrile, toluene, benzene, ethanol, methanol or water at 0° C. to 100° C. with the preferred conditions being sodium triacetoxyborohydride in 1,2-dichloroethane at 25° C. to 90° C. to afford a compound of formula I(b).

Also referring to Scheme 2, a compound of formula VIII can be reacted with a compound of formula X in which R⁶ is as defined above, except that R⁶ may not be hydrogen, and L is a leaving group (e.g., Cl, Br, I, OSO₂ alkyl, OSO₂aryl) in the presence or absence of base (e.g., sodium or potassium hydroxide, sodium or potassium or cesium carbonate, sodium or potassium tert-butoxide, sodium or potassium hydrogen carbonate, sodium or potassium acetate) in the presence or absence of an inert reaction solvent such as water, methanol, ethanol, isopropanol, acetonitrile, methylene chloride, chloroform, 1,2-dichloroethane, tetrahydrofuran, diethylether, dioxane, 1,2-dimethoxyethane, benzene, toluene, dimethylformamide, or dimethylsulfoxide at a temperature from about −10° C. to about 150° C. to produce a compound of formula I(c). The preferred conditions are L=Br, in ethanol at 25° C. to 78° C.

Also referring to Scheme 2, a compound of formula VIII can undergo a transformation to replace a diazonium group derived from an aryl amine with a fluorine to yield a compound of formula I(d). The most commonly used procedure for diazotization of an aryl amine involves sodium nitrite in aqueous hydrochloric acid or sulfuric acid. Fluoro-containing counter ions may be then introduced into the reaction mixture to convert the diazonium ion to a fluorine. Commonly used counter-ions include, but not limited to BF₄⁻, PF₆⁻, AsF6⁻ and SbF₆⁻. Hydrogen fluoride may also be used as a fluoride source to prepare a compound of formula I(d). For a review of this transformation, see: H. Zollinger, Diazo Chemistry I, VCH, Weinheim, 1994 (Chapter 10).

The compounds of the formula I and their pharmaceutically acceptable salts (hereafter “the active compounds”) can be administered via either the oral, transdermal (e.g., through the use of a patch), intranasal, sublingual, rectal, parenteral or topical routes. Transdermal and oral administration are preferred. These compounds are, most desirably, administered in dosages ranging from about 0.25 mg up to about 1500 mg per day, preferably from about 0.25 to about 300 mg per day in single or divided doses, although variations will necessarily occur depending upon the weight and condition of the subject being treated and the particular route of administration chosen. However, a dosage level that is in the range of about 0.01 mg to about 10 mg per kg of body weight per day is most desirably employed. Variations may nevertheless occur depending upon the weight and condition of the persons being treated and their individual responses to said medicament, as well as on the type of pharmaceutical formulation chosen and the time period and interval during which such administration is carried out. In some instances, dosage levels below the lower limit of the aforesaid range may be more than adequate, while in other cases still larger doses may be employed without causing any harmful side effects, provided that such larger doses are first divided into several small doses for administration throughout the day.

The active compounds can be administered alone or in combination with pharmaceutically acceptable carriers or diluents by any of the several routes previously indicated. More particularly, the active compounds can be administered in a wide variety of different dosage forms, e.g., they may be combined with various pharmaceutically acceptable inert carriers in the form of tablets, capsules, transdermal patches, lozenges, troches, hard candies, powders, sprays, creams, salves, suppositories, jellies, gels, pastes, lotions, ointments, aqueous suspensions, injectable solutions, elixirs, syrups, and the like. Such carriers include solid diluents or fillers, sterile aqueous media and various non-toxic organic solvents. In addition, oral pharmaceutical compositions can be suitably sweetened and/or flavored. In general, the active compounds are present in such dosage forms at concentration levels ranging from about 5.0% to about 70% by weight.

For oral administration, tablets containing various excipients such as microcrystalline cellulose, sodium citrate, calcium carbonate, dicalcium phosphate and glycine may be employed along with various disintegrants such as starch (preferably corn, potato or tapioca starch), alginic acid and certain complex silicates, together with granulation binders like polyvinylpyrrolidone, sucrose, gelatin and acacia. Additionally, lubricating agents such as magnesium stearate, sodium lauryl sulfate and talc can be used for tabletting purposes. Solid compositions of a similar type may also be employed as fillers in gelatin capsules; preferred materials in this connection also include lactose or milk sugar, as well as high molecular weight polyethylene glycols. When aqueous suspensions and/or elixirs are desired for oral administration the active ingredient may be combined with various sweetening or flavoring agents, coloring matter and, if so desired, emulsifying and/or suspending agents, together with such diluents as water, ethanol, propylene glycol, glycerin and various combinations thereof.

For parenteral administration, a solution of an active compound in either sesame or peanut oil or in aqueous propylene glycol can be employed. The aqueous solutions should be suitably buffered (preferably pH greater than 8), if necessary, and the liquid diluent first rendered isotonic. These aqueous solutions are suitable for intravenous injection purposes. The oily solutions are suitable for intraarticular, intramuscular and subcutaneous injection purposes. The preparation of all these solutions under sterile conditions is readily accomplished by standard pharmaceutical techniques well known to those skilled in the art.

It is also possible to administer the active compounds topically and this can be done by way of creams, a patch, jellies, gels, pastes, ointments and the like, in accordance with standard pharmaceutical practice.

The compounds of the invention show advantageous potency as measured by functional activation of the α7/5-HT₃ chimeric receptor, or high selectivity over other ion channels, such as 5-HT₃ or the IKr channel, or a combination thereof. The high selectivity over other ion channels, such as 5-HT₃ and/or the IKr channel, is an exemplary advantage of the compounds of the invention.

The effectiveness of the active compounds in suppressing nicotine binding to specific receptor sites can be determined by the following procedure, which is a modification of the methods of Lippiello, P. M. and Femandes, K. G. (in “The Binding of L-[³H]Nicotine To A Single Class of High-Affinity Sites in Rat Brain Membranes”, Molecular Pharm., 29, 448-54, (1986)) and Anderson, D. J. and Arneric, S. P. (in “Nicotinic Receptor Binding of ³H-Cystisine, ³H-Nicotine and ³H-Methylcarmbamylcholine In Rat Brain”, European J. Pharm., 253, 261-67 (1994)). Male Sprague-Dawley rats (200-300 g) from Charles River were housed in groups in hanging stainless steel wire cages and were maintained on a 12 hour light/dark cycle (7 a.m.-7 p.m. light period). They received standard Purina Rat Chow and water ad libitum. The rats were killed by decapitation. Brains were removed immediately following decapitation. Membranes were prepared from brain tissue according to the methods of Lippiello and Fernandez (Molec. Pharmacol., 29, 448-454, (1986)) with some modifications. Whole brains were removed, rinsed with ice-cold buffer, and homogenized at 0° in 10 volumes of buffer (w/v) using a Brinkmann Polytron™ (Brinkmann Instruments Inc., Westbury, N.Y.), setting 6, for 30 seconds. The buffer consisted of 50 mM Tris HCl at a pH of 7.5 at room temperature. The homogenate was sedimented by centrifugation (10 minutes; 50,000×g; 0° to 4° C.). The supernatant was poured off and the membranes were gently resuspended with the Polytron and centrifuged again (10 minutes; 50,000×g; 0° C. to 4° C.). After the second centrifugation, the membranes were resuspended in assay buffer at a concentration of 1.0 g/100 mL. The composition of the standard assay buffer was 50 mM Tris HCl, 120 mM NaCl, 5 mM KCl, 2 mM MgCl₂, 2 mM CaCl₂ and had a pH of 7.4 at room temperature.

Routine assays were performed in borosilicate glass test tubes. The assay mixture typically consisted of 0.9 mg of membrane protein in a final incubation volume of 1.0 mL. Three sets of tubes were prepared wherein the tubes in each set contained 50 μL of vehicle, blank, or test compound solution, respectively. To each tube was added 200 μL of [3H]-nicotine in assay buffer followed by 750 μL of the membrane suspension. The final concentration of nicotine in each tube was 0.9 nM. The final concentration of cytisine in the blank was 1 μM. The vehicle consisted of deionized water containing 30 μL of 1 N acetic acid per 50 mL of water. The test compounds and cytisine were dissolved in vehicle. Assays were initiated by vortexing after addition of the membrane suspension to the tube. The samples were incubated at 0° to 4° C. in an iced shaking water bath. Incubations were terminated by rapid filtration under vacuum through Whatman GF/B™ glass fiber filters (Brandel Biomedical Research & Development Laboratories, Inc., Gaithersburg, Md.) using a Brandel™ multi-manifold tissue harvester (Brandel Biomedical Research & Development Laboratories, Inc., Gaithersburg, Md.). Following the initial filtration of the assay mixture, filters were washed two times with ice-cold assay buffer (5 ml each). The filters were then placed in counting vials and mixed vigorously with 20 ml of Ready Safe™ (Beckman, Fullerton, Calif.) before quantification of radioactivity. Samples were counted in a LKB Wallac Rackbeta™ liquid scintillation counter (Wallac Inc., Gaithersburg, Md.) at 40-50% efficiency. All determinations were in triplicate.

Calculations: Specific binding (C) to the membrane is the difference between total binding in the samples containing vehicle only and membrane (A) and non-specific binding in the samples containing the membrane and cytisine (B), i.e.,
Specific binding=(C)=(A)−(B).

Specific binding in the presence of the test compound (E) is the difference between the total binding in the presence of the test compound (D) and non-specific binding (B), i.e., (E)=(D)−(B).
% Inhibition=(1−((E)/(C))times 100.

The compounds of the invention that were tested in the above assay preferably exhibit IC₅₀ values of less than 10 μM.

[¹²⁵I]-Bungarotoxin Binding to α7 Nicotinic Receptors in GH₄Cl Cells:

Membrane preparations were made for nicotinic receptors expressed in GH₄Cl cell line. Briefly, one gram of cells by wet weight were homogenized with a polytron in 25 mis of buffer containing 20 mM Hepes, 118 mM NaCl, 4.5 mM KCl, 2.5 mM CaCl₂, 1.2 mM MgSO₄, pH 7.5. The homogenate was centrifuged at 40,000×g for 10 min at 4° C., the resulting pellet was homogenized and centrifuged again as described above. The final pellet was resuspended in 20 mis of the same buffer. Radioligand binding was carried out with [¹²⁵I] alpha-bungarotoxin from New England Nuclear, specific activity about 16 uCi/ug, used at 0.4 nM final concentration in a 96 well microtiter plate. The plates were incubated at 37° C. for 2 hours with 25 μl drugs or vehicle for total binding, 100 μl [¹²⁵I] Bungarotoxin and 125 μl tissue preparation. Nonspecific binding was determined in the presence of methyllycaconitine at 1 μM final concentration. The reaction was terminated by filtration using 0.5% Polyethylene imine treated Whatman GF/B™ glass fiberfilters (Brandel Biomedical Research & Development Laboratories, Inc., Gaithersburg, Md.) on a Skatron cell harvester (Molecular Devices Corporation, Sunnyvale, Calif.) with ice-cold buffer, filters were dried overnight, and counted on a Beta plate counter using Betaplate Scint. (Wallac Inc., Gaithersburg, Md.). Data are expressed as IC50's (concentration that inhibits 50% of the specific binding) or as an apparent Ki, IC50/1+[L]/KD. [L]=ligand concentration, KD=affinity constant for [¹²⁵I] ligand determined in separate experiment.

The compounds of the invention that were tested in the above assay preferably exhibit IC₅₀ values of less than 10 μM.

[¹²⁵I]-Bungarotoxin Binding to Alpha1 Nicotinic Receptors in Torpedo Electroplax Membranes:

Frozen Torpedo electroplax membranes (100 μl) were resuspended in 213 mis of buffer containing 20 mM Hepes, 118 mM NaCl, 4.5 mM KCl, 2.5 mM CaCl₂, 1.2 mM MgSO₄, pH 7.5 with 2 mg/ml BSA. Radioligand binding was carried out with [¹²⁵I] alpha-bungarotoxin from New England Nuclear, specific activity about 16 uCi/ug, used at 0.4 nM final concentration in a 96 well microtiter plate. The plates were incubated at 37° C. for 3 hours with 25 μl drugs or vehicle for total binding, 100 μl [¹²⁵I] Bungarotoxin and 125 μl tissue preparation. Nonspecific binding was determined in the presence of alpha-bungarotoxin at 1 μM final concentration. The reaction was terminated by filtration using 0.5% Polyethylene imine treated GF/B filters on a Brandel cell harvester with ice-cold buffer, filters were dried overnight, and counted on a Beta plate counter using Betaplate Scint. Data are expressed as IC50's (concentration that inhibits 50% of the specific binding) or as an apparent Ki, IC50/1+[L]/KD. [L]=ligand concentration, KD=affinity constant for [¹²⁵I] ligand determined in separate experiment.

The compounds of the invention that were tested in the above assay preferably exhibit IC₅₀ values of greater than 10 nM, more preferably greater than 100 nM.

5-HT₃ Receptor Binding in NG-108 Cells Using 3H-LY278584:

NG-108 cells endogenously express 5-HT₃ receptors. Cells are grown in DMEM containing 10% fetal bovine serum supplemented with L-glutamine (1:100). Cells are grown to confluence and harvested by removing the media, rinsing the flasks with phosphate buffered saline (PBS) and then allowed to sit for a 2-3 minutes with PBS containing 5 mM EDTA. Cells are dislodged and poured into a centrifuge tube. Flasks are rinsed with PBS and added to centrifuge tube. The cells are centrifuged for ten minutes at 40,000×g (20,000 rpm in Sorvall SS34 rotor (Kendro Laboratory Products, Newtown, Conn.)). The supernatant is discarded (into chlorox) and at this point the remaining pellet is weighed and can be stored frozen (−80 degrees C.) until used in the binding assay. Pellets (fresh or frozen −250 mgs per 96 well plate) are homogenized in 50 mM Tris HCl buffer containing 2 mM MgCl₂ (pH 7.4) using a Polytron homogenizer (setting 15,000 rpm) for ten seconds. The homogenate is centrifuged for ten minutes at 40,000×g. The supernatant is discarded and the pellet resuspended with the Polytron in fresh ice-cold 50 mM Tris HCl containing 2 mM MgCl₂ (pH 7.4) buffer and centrifuged again. The final pellet is resuspended in assay buffer (50 mM Tris HCl buffer (pH 7.4 at 37° C. degrees) containing 154 mM NaCl,) for a final tissue concentration of 12.5 mg per mL buffer (1.25× final concentration). Incubations were initiated by the addition of tissue homogenate to 96 well polypropylene plates containing test compounds that have been diluted in 10% DMSO/50 mM Tris buffer and radioligand (1 nM final concentration of 3H-LY278584). Nonspecific binding was determined using a saturating concentration of a known potent 5-HT₃ antagonist (10 μM ICS-205930). After an hour incubation at 37° C. in a water bath, the incubation is ended by rapid filtration under vacuum through a fire-treated Whatman GF/B glass fiber filter (presoaked in 0.5% Polyethylene imine for two hours and dried) using a 96 well Skatron Harvester (3 sec pre-wet; 20 seconds wash; 15 seconds dry). Filters are dried overnight and then placed into Wallac sample bags with 10 mLs BetaScint. Radioactivity is quantified by liquid scintillation counting using a BetaPlate counter (Wallac, Gaithersburg, Md.). The percent inhibition of specific binding is calculated for each concentration of test compound. An IC50 value (the concentration which inhibits 50% of the specific binding) is determined by linear regression of the concentration-response data (log concentration vs. logit percent values). Ki values are calculated according to Cheng & Prusoff −Ki=IC50/(1+(L/Kd)), where L is the concentration of the radioligand used in the experiment and the Kd value is the dissociation constant for the radioligand determined in separate saturation experiments.

The compounds of the invention that were tested in the above assay preferably exhibit IC50 values of greater than 10 nM, more preferably greater than 100 nM.

### Cell-Based Assay for Measuring the EC₅₀ of α7 nAChR Agonists

Construction and Expression of the α7-5HT₃ Receptor:

The cDNA encoding the N-terminal 201 amino acids from the human α7 nAChR that contain the ligand binding domain of the ion channel was fused to the cDNA encoding the pore forming region of the mouse 5HT₃ receptor as described by Eisele J L, et al., “Chimaeric nicotinic-serotonergic receptor combines distinct ligand binding and channel specificities,” Nature (1993), Dec. 2; 366(6454):479-83, and modified by Groppi, et al., WO 00/73431. The chimeric α7-5HT₃ ion channel was inserted into pGS175 and pGS179 which contain the resistance genes for G-418 and hygromycin B, respectively. Both plasmids were simultaneously transfected into SH-EP1 cells and cell lines were selected that were resistant to both G-418 and hyrgromycin B. Cell lines expressing the chimeric ion channel were identified by their ability to bind fluorescent α-bungarotoxin on their cell surface. The cells with the highest amount of fluorescent α-bungarotoxin binding were isolated using a Fluorescent Activated Cell Sorter (FACS). Cell lines that stably expressed the chimeric α7-5HT₃ were identified by measuring fluorescent α-bungarotoxin binding after growing the cells in minimal essential medium containing nonessential amino acids supplemented with 10% fetal bovine serum, L-glutamine, 100 units/ml penicillin/streptomycin, 250 ng/mg fungizone, 400 μg/ml hygromycin B, and 400 μg/ml G-418 at 37° C. with 6% CO₂ in a standard mammalian cell incubator for at least 4 weeks in continuous culture.

### Assay of the Activity of the Chimeric α7-5HT₃ Receptor

To assay the activity of the α7-5HT₃ ion channel, cells expressing the channel were plated into each well of either a 96 or 384 well dish (Corning #3614) and grown to confluence prior to assay. On the day of the assay, the cells were loaded with a 1:1 mixture of 2 mM Calcium Green 1, AM (Molecular Probes) dissolved in anhydrous DMSO and 20% pluronic F-127 (Molecular Probes). This solution was added directly to the growth media of each well to achieve a final concentration 2 μM. The cells were incubated with the dye for 60 min at 37° C. and is washed with a modified version of Earle's balanced salt solution (MMEBSS) as described in WO 00/73431. The ion conditions of the MMEBSS was adjusted to maximize the flux of calcium ion through the chimeric α7-5HT₃ ion channel as described in WO 00/73431. The activity of compounds on the chimeric α7-5HT₃ ion channel was analyzed on FLIPR. The instrument was set up with an excitation wavelength of 488 nanometers using 500 milliwatts of power. Fluorescent emission was measured above 525 nanometers with an appropriate F-stop to maintain a maximal signal to noise ratio. Agonist activity of each compound was measured by directly adding the compound to cells expressing the chimeric α7-5HT₃ ion channel and measuring the resulting increase in intracellular calcium that is caused by the agonist-induced activation of the chimeric ion channel. The assay is quantitative such that concentration-dependent increase in intracelluar calcium is measured as concentration-dependent change in Calcium Green fluorescence. The effective concentration needed for a compound to cause a 50% maximal increase in intracellular calcium is termed the EC₅₀.

The compounds of the invention that were tested in the above assay preferably exhibit IC₅₀ values of less than 10 μM, more preferably less than 1 μM.

The following experimental examples illustrate but do not limit the present invention. In the examples, commercial reagents were used without further purification. Purification by chromatography was done on prepacked silica columns from Biotage (Dyax Corp, Biotage Division, Charlottesville, Va.). Melting points (mp) were obtained using a Mettler Toledo FP62 melting point apparatus (Mettler-Toledo, Inc., Worthington, Ohio) with a temperature ramp rate of 10° C./min and are uncorrected. Proton nuclear magnetic resonance (¹H NMR) spectra were recorded in deuterated solvents on a Varian INOVA400 (400 MHz) spectrometer (Varian NMR Systems, Palo Alto, Calif.). Chemical shifts are reported in parts per million (ppm, δ) relative to Me₄Si (δ 0.00). Carbon-13 nuclear magnetic resonance (¹³C NMR) spectra were recorded on a Varian INOVA400 (100 MHz). Chemical shifts are reported in ppm (δ) relative to the central line of the 1:1:1 triplet of deuterochloroform (δ 77.00), the center line of deuteromethanol (δ 49.0) or deuterodimethylsulfoxide (δ 39.7). The number of carbon resonances reported may not match the actual number of carbons in some molecules due to magnetically and chemically equivalent carbons and may exceed the number of actual carbons due to conformational isomers. Mass spectra (MS) were obtained using a Waters ZMD mass spectrometer using flow injection atmospheric pressure chemical ionization (APCI) (Waters Corporation, Milford, Mass.). Gas chromatography with mass detection (GCMS) were obtained using a Hewlett Packard HP 6890 series GC system with a HP 5973 mass selective detector and a HP-1 (crosslinked methyl siloxane) column (Agilent Technologies, Wilmington, Del.). LC-MS spectra were recorded on a Water ZQ 1525μ Mass Spectrometry with Electrospray (ESI+) and a Binary HPLC Pump at 25° C. using gradient elution. Solvent A is 98% water, 2% acetonitrile with 0.01% formic acid, Solvent B is 100% acetonitrile with 0.005% formic acid. A linear gradient over 3.55 min was used starting at 95% A, 5% B and ending at 0% A, 100% B with a flow rate of 1 mL/min. Room temperature (RT) refers to 20-25° C. The abbreviations “h” and “hrs” refer to “hours”. 1,4-Diaza-bicyclo[3.2.2]nonane was prepared via slight modifications of the published procedure: see, Rubstov, M. V.; Mikhlina, E. E.; Vorob'eva, V. Ya.; Yanina, A. Zh. Obshch. Khim. 1964, V34, 2222-2226.

### EXAMPLES

### Example 1

### 4-(6-Bromo-5-methyloxazolo[4,5-b]pyridin-2-yl)-1,4-diaza-bicyclo[3.2.2]nonane

A flask, equipped with a magnetic stirring bar, was charged with 4-(5-methyloxazolo[4,5-b]pyridin-2-yl)-1,4-diaza-bicyclo[3.2.2]nonane (1.0 g, 3.02 mmol), as described in EP 1219622 A2, sodium acetate (3.15 g, 36.1 mmol), and 50% AcOH aqueous solution (40 mL). The flask was purged with nitrogen and closed and the mixture was stirred at room temperature to form a homogeneous solution. Then/Br₂ (170 uL, 3.2 mmol) was added dropwise and the mixture was stirred for 15 min (LCMS showed incomplete conversion). Additional bromine (75 uL, 1.45 mmol) was added and stirred 10 min (LCMS showed starting material was gone). The mixture was cooled with an ice bath and basified with 12 N NaOH to pH 14. The mixture was then extracted with 5% CH₂Cl₂ in methanol three times and the extract was dried over MgSO₄ and evaporated to give 720 mg of 4-(6-bromo-5-methyloxazolo[4,5-b]pyridin-2-yl)-1,4-diaza-bicyclo[3.2.2]nonane. Yield 71%. The product was dissolved in MeOH, then 5 mL of 4M HCl in dioxane was added, and the solvent was removed under vacuum. The resulted residue was re-crystallized from methanol and diethyl ether to afford the HCl salt. MS for C₁₄H₁₇BrN₄O m/z 337.2 (M+H)⁺.

Following the same procedure from 4-(5-ethyl-oxazolo[4,5-b]pyridin-2-yl)-1,4-diaza-bicyclo[3.2.2]nonane, the following example was synthesized:

### Example 2

### 4-(6-Bromo-5-ethyl-oxazolo[4,5-b]pyridin-2-yl)-1,4-diaza-bicyclo[3.2.2]nonane

LC-MS for C₁₅H₁₉BrN₄O: retention time 1.4 min, m/z 353.0 (M+H)⁺.

### Example 3

### 4-(5,6-Dimethyloxazolo[4,5-b]pyridin-2-yl)-1,4-diaza-bicyclo[3.2.2]nonane

A microwave reactor tube (Smith Process Vial), equipped with a magnetic stirring bar, was charged under nitrogen with 4-(6-bromo-5-methyloxazolo[4,5-b]pyridin-2-yl)-1,4-diaza-bicyclo[3.2.2]nonane hydrochloride (100 mg, 0.3 mmol), Pd(dppf)₂Cl₂.CH₂Cl₂ (5 mg, 0.006 mmol), 2 mL of anhydrous dioxane, and ZnMe₂ (0.3 mL of 2M solution in toluene, 0.6 mmol). The vial was flushed with nitrogen, sealed, and heated to 150° C. for 15 minutes in a microwave reactor (Smithcreator of Personal Chemistry). The mixture was diluted with 3 mL of MeOH, filtered through celite, and celite cake was washed with 3 mL of MeOH. The clear solution was evaporated and the residue was purified by flash chromatography (silica gel, 5% to 10% MeOH in CH₂Cl₂ with 1% NH₄OH) to give 4-(5,6-dimethyloxazolo[4,5-b]pyridin-2-yl)-1,4-diaza-bicyclo[3.2.2]nonane. The product was dissolved in 0.5 mL of MeOH, then 0.5 mL of 2M HCl in ether was added, and the mixture was allowed to crystallize. The precipitate was collected by filtration and dried under vacuum to give 4-(5,6-dimethyloxazolo[4,5-b]pyridin-2-yl)-1,4-diaza-bicyclo[3.2.2]nonane. Yield 58%. MS for C₁₅H₂₀N₄O m/z 273.3 (M+H)⁺.

Following the procedure from 4-(6-Bromo-5-ethyl-oxazolo[4,5-b]pyridin-2-yl)-1,4-diaza-bicyclo[3.2.2]nonane, the following example was synthesized:

### Example 4

### 4-(6-Methyl-5-ethyl-oxazolo[4,5-b]pyridin-2-yl)-1,4-diaza-bicyclo[3.2.2]nonane

LC-MS for C₁₆H₂₂N₄O: retention time 0.6 min, m/z 287.2 (M+H)⁺.

### Example 5

### 4-(5-Methyl-6-nitro-oxazolo[4,5-b]pyridin-2-yl)-1,4-diaza-bicyclo[3.2.2]nonane

4-(5-Methyl-oxazolo[4,5-b]pyridin-2-yl)-1,4-diaza-bicyclo[3.2.2]nonane (0.6 g) was dissolved in sulfuric acid (95-98%, 4.0 mL), cooled to 0° C. in an ice bath. A chilled mixture of sulfuric acid (95-98%, 2.0 mL) and nitric acid (>90%, 2 mL) was added slowly and the resulted mixture was allowed to stir at ambient temperature for 16 hours, and then slowly poured over NaHCO₃ (15.0 g). A solution of NaOH (1.0 N aqueous solution) was added to adjust the pH to −14. The mixture was then extracted with CH₂Cl₂ (3×50 mL). The combined organic layers were dried over Na₂SO₄. The solvent was removed in vacuo, and the residue was purified using flash chromatography (silica gel, 0% to 9.5% MeOH in CH₂Cl₂ with 0.5% NH₄OH). Yield 33%. MS for C₁₄H₁₇NSO₃ m/z 304.3 (M+H)⁺.

### Example 6

### 2-(1,4-Diaza-bicyclo[3.2.2]non-4-yl)-5-methyl-oxazolo[4,5-b]pyridin-6-ylamine

To a solution of 4-(5-Methyl-6-nitro-oxazolo[4,5-b]pyridin-2-yl)-1,4-diaza-bicyclo[3.2.2]nonane (0.208 g) in 1:1 EtOH/MeOH (100 mL) was added 10% Pd/C (0.050 g). The mixture was shaken under H₂ (45 psi) for 2 hours in a PARR apparatus, filtered through a pad of celite and the filtrate was concentrated to give 2-(1,4-Diaza-bicyclo[3.2.2]non-4-yl)-5-methyl-oxazolo[4,5-b]pyridin-6-ylamine. MS for C₁₄H₁₉N₅O m/z 274.3 (M+H)⁺.

### Example 7

### 4-(6-Fluoro-5-methyl-oxazolo[4,5-b]pyridin-2-yl)-1,4-diaza-bicyclo[3.2.2]nonane

2-(1,4-Diaza-bicyclo[3.2.2]non-4-yl)-5-methyl-oxazolo[4,5-b]pyridin-6-ylamine (0.180 g) was dissolved in a solution of HCl (36%, 0.4 mL) and water (2 mL). The resulted mixture was heated to 100° C. for 20 minutes and then cooled to −10° C. in an ice-NaCl bath. A solution of NaNO₂ (0.055 g) in water (2 mL) was added, followed by the addition of HPF₆ (60%, 0.17 mL). The resulted suspension was stirred at −10° C. for additional 30 minutes. The mixture was then filtered to give a solid, which was transferred to a vial and heated to 165° C. in an oil bath for 20 minutes. The residue was purified using reversed phase HPLC. Yield 6%. MS for C₁₄H₁₇FN₄O m/z 277.3 (M+H)⁺.

### Example 8

### 4-(6-Chloro-5-methyl-oxazolo[4,5-b]pyridin-2-yl)-1,4-diaza-bicyclo[3.2.2]nonane

4-(5-Methyl-oxazolo[4,5-b]pyridin-2-yl)-1,4-diaza-bicyclo[3.2.2]nonane (0.1 g), N-chlorosuccinimide (0.051 g) were dissolved in CHCl₃, sealed in a microwave reactor tube (Smith Process Vial), and heated to 150° C. for 10 minutes in a microwave reactor (Smithcreator of Personal Chemistry). The mixture was filtered and the solvent was removed in vacuo. The residue was dissolved in MeOH and HCl in 1,4-dioxane (4 M, 0.4 mL) was added. The solvent was removed in vacuo and the residue was dissolved in MeOH and triturated with CH₂Cl₂ to give a solid. Yield: 70.3%. MS for C₁₄H₁₇ClN₄O m/z 293.0 (M+H)⁺.

Following the same procedure from 4-(5-Ethyl-oxazolo[4,5-b]pyridin-2-yl)-1,4-diaza-bicyclo[3.2.2]nonane, the following example was synthesized:

### Example 9

### 4-(6-Chloro-5-ethyl-oxazolo[4,5-b]pyridin-2-yl)-1,4-diaza-bicyclo[3.2.2]nonane

LC-MS for C₁₅H₂₀ClN₄O: retention time 1.4 min, m/z 307.1 (M+H)⁺.

### Example 10

### 4-(5-methyl-6-phenyloxazolo[4,5-b]pyridin-2-yl)-1,4-diaza-bicyclo[3.2.2]nonane

4-(6-Bromo-5-methyl-oxazolo[4,5-b]pyridin-2-yl)-1,4-diaza-bicyclo[3.2.2]nonane (50 mg, 0.15 mmol), phenyl boronic acid (20 mg), tetrakis(triphenylphosphine)palladium (0) (9 mg), potassium carbonate (82 mg), ethanol (1 mL) and H₂O (0.10 mL) were combined in a microwave reactor tube (Smith Process Vial) equipped with a stirring bar. The container was purged with nitrogen, sealed in and heated to 100° C. for 8 minutes in a microwave reactor (Smithcreator of Personal Chemistry). The mixture was extracted with 5% methanol in CH₂Cl₂. The organic layer was dried over MgSO₄, and concentrated in vacuo. The residue was purified using flash chromatography (silica gel, 7% MeOH in CH₂Cl₂). The product was dissolved in 0.5 mL of MeOH, then 0.5 mL of 4M HCl in dioxane was added, and the solvent was removed under vacuum. The resulted residue was re-crystallized from methanol and diethyl ether. Yield 33%. MS for C₂₀H₂₂N₄O m/z 335.3 (M+H)⁺.

### Example 11

### 4-(5-Methyl-6-phenoxyoxazolo[4,5-b]pyridin-2-yl)-1,4-diaza-bicyclo[3.2.2]nonane

4-(6-Bromo-5-methyl-oxazolo[4,5-b]pyridin-2-yl)-1,4-diaza-bicyclo[3.2.2]nonane (100 mg, 0.3 mmol), phenol (62 mg), CuCl (15 mg), tetramethyl heptane 3,5-dione (5 mg, 0.03 mmol), cesium carbonate (193 mg, 0.6 mmol), and NMP (2 mL) was sealed in a microwave reactor tube (Smith Process Vial) equipped with a stirring bar, purged with nitrogen, and heated to 200° C. for 10 minutes in a microwave reactor (Smithcreator of Personal Chemistry). The residue was dissolved in MeOH and filtered through a pad of celite. The cake was further washed with MeOH. The filtrate was concentrated in vacuo and the residue was purified using flash chromatography (silica gel, 7% MeOH in CH₂Cl₂). The product was dissolved in 0.5 mL of MeOH, then 0.5 mL of 4M HCl in dioxane was added, and solvent was removed under vacuum. The resulted residue was re-crystallized from methanol and diethyl ether. Yield: 4%. MS for C₂₀H₂₂N₄O₂ m/z 351.3 (M+H)⁺.

### Example 12

### 2-(1,4-Diaza-bicyclo[3.2.2]nonan-4-yl)-5-methyloxazolo[4,5-b]pyridine-6-carbonitrile

4-(6-Bromo-5-methyl-oxazolo[4,5-b]pyridin-2-yl)-1,4-diaza-bicyclo[3.2.2]nonane (0.08 g), CuCN (0.133 g) were dissolved in DMF (1 mL). The resulted suspension was sealed in a tube (Smith Process Vial) and heated to 250° C. for 10 minutes in a microwave reactor (Smithcreator of Personal Chemistry). The mixture was filtered and the filtrate was concentrated in vacuo. The residue was purified using reversed phase HPLC. Yield 6%. MS for C₁₅H₁₇N₅O m/z 284.3 (M+H)⁺.

1. A compound of the formula I 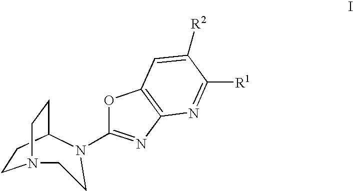
wherein
R¹ is selected from the group consisting of —CN, (C₁-C₈) alkyl, (C₃-C₈) cycloalkyl, 3-8 membered heterocycloalkyl, (C₆-C₁₀) aryl, -5-12 membered heteroaryl, OR³, —C(═O)NR³R⁴, —NR³C(═O)R⁴, —S(═O)₂R³, —S(═O)₂NR³R⁴—NR³R⁴, wherein each said alkyl, cycloalkyl, heterocycloalkyl, aryl and heteroaryl is optionally substituted with one or more substituents independently selected from F, Cl, Br, I, nitro, CN, CF₃, —NR⁹R¹⁰, —NR⁹C(═O)R¹⁰, —OR⁹, —C(═O)OR⁹, —C(═O)R⁹, —C(═O)NR⁹R¹⁰, —S(═O)₂NR⁹R¹⁰ and R⁹;
R² is selected from the group consisting of F, Cl, Br, I, nitro, —CN, CF₃, (C₁-C₈)alkyl, (C₃-C₈)cycloalkyl, 3-8 membered heterocycloalkyl, (C₆-C₁₀) aryl, and 5-12 membered heteroaryl, —NR⁶R⁷, —NR⁶C(═O)R⁷, —NR⁶S(═O)₂R⁷, —OR⁶, —OC(═O)R⁶, —C(═O)OR⁶, —C(═O)R⁶, —C(═O)NR⁶R⁷, —SR⁶, —S(═O)R⁶, —S(═O)₂R⁶, —S(═O)₂NR⁶R⁷; wherein each said alkyl, cycloalkyl, heterocycloalkyl, aryl and heteroaryl is optionally substituted with one or more substituents independently selected from F, Cl, Br, I, nitro, CN, CF₃, —NR⁹R¹⁰, —NR⁹C(═O)R¹⁰, —OR⁹, —C(═O)OR⁹, —C(═O)R⁹, —C(═O)NR⁹R¹⁰, —SR⁹, —S(═O)R⁹, —S(═O)₂R⁹, —S(═O)₂NR⁹R¹⁰ and R⁹;
each of R³, R⁴, R⁶ and R⁷ is independently selected from H, straight chain or branched (C₁-C₈)alkyl, (C₃-C₈)cycloalkyl, 3-8 membered heterocycloalkyl, (C₆-C₁₀) aryl, and 5-12 membered heteroaryl; wherein each of R³, R⁴, R⁶ and R⁷ is optionally substituted with one to six substituents independently selected from F, Cl, Br, I, nitro, cyano, CF₃, —NR⁹R¹⁰, —NR⁹C(═O)R¹⁰, —NR⁹S(═O)₂R¹⁰, —OR⁹, —OC(═O)R⁹, —C(═O)OR⁹, —C(═O)R⁹, —C(═O)NR⁹R¹⁰, —SR⁹, —S(═O)R⁹, —S(═O)₂R⁹, —S(═O)₂NR⁹R¹⁰ and R⁹;
or R³ and R⁴ taken together with the nitrogen of NR³R⁴ form a 3-8 membered heterocycloalkyl;
or R⁶ and R⁷ taken together with the nitrogen of NR⁶R⁷ form a 3-8 membered heterocycloalkyl;
each of R⁹ and R¹⁰ is independently selected from H, straight chain or branched (C₁-C₈)alkyl, (C₃-C₈)cycloalkyl, 3-8 membered heterocycloalkyl, (C₆-C₁₀) aryl or 5-12 membered heteroaryl; wherein each of R⁹ and R¹⁰ is optionally substituted with one to six substituents independently selected from F, Cl, Br, I, nitro, cyano, CF₃, —NR¹²R¹³, —NR¹²C(═O)R¹³, —NR¹²S(═O)₂R¹³, —OR¹², —C(═O)NR¹²R¹³, —SR¹², —S(═O)R¹², —S(═O)₂R¹², —S(═O)₂NR¹²R¹³ and R¹²;
or R⁹ and R¹⁰ taken together with the nitrogen of NR⁹R¹⁰ form a 3-8 membered heterocycloalkyl;
each of R¹² and R¹³ is independently selected from H, straight chain or branched (C₁-C₈)alkyl, (C₃-C₈)cycloalkyl, 3-8 membered heterocycloalkyl, (C₆-C₁₀) aryl and (5-12 membered) heteroaryl;
or a pharmaceutically acceptable salt thereof.

2. The compound of claim 1 wherein R¹ is (C₁-C₈) alkyl, (C₃-C₈) cycloalkyl, 3-8 membered heterocycloalkyl, (C₆-C₁₀) aryl, -5-12 membered heteroaryl, wherein each said alkyl, cycloalkyl, heterocycloalkyl, aryl and heteroaryl is optionally substituted with one or more substituents independently selected from F, Cl, Br, I, nitro, CN, CF₃, —NR⁹R¹⁰, —OR⁹, and R⁹.

3. The compound of claim 1 wherein R¹ is (C₁-C₈) alkyl wherein said alkyl is optionally substituted with one or more substituents independently selected from the group consisting of F, Cl, Br, I, nitro, CN, CF₃, —NR⁹R¹⁰, —OR⁹, and R⁹.

4. The compound of claim 1 wherein R¹ is (C₆-C₁₀) aryl or 5-12 membered heteroaryl, wherein each of said aryl and heteroaryl is optionally substituted with one or more substituents independently selected from the group consisting of F, Cl, Br, I, nitro, CN, CF₃, —NR⁹R¹⁰, —OR⁹, and R⁹.

5. The compound of claim 3 wherein R¹ is unsubstituted C₁-C₈ alkyl.

6. The compound of claim 1 wherein R is selected from the group consisting of F, Cl, Br, I, nitro, —CN, CF₃, (C₁-C₈)alkyl, (C₆-C₁₀) aryl, and 5-12 membered heteroaryl, —NR⁶R⁷, —OR⁶ wherein each said alkyl, cycloalkyl, heterocycloalkyl, aryl and heteroaryl is optionally substituted with one or more substituents independently selected from F, Cl, Br, I, nitro, CN, CF₃, —NR⁹R¹⁰, —NR⁹C(═O)R¹⁰, —OR⁹, —C(═O)OR⁹, —C(═O)R⁹, —C(═O)NR⁹R¹⁰, —SR⁹, —S(═O)R⁹, —S(═O)₂R⁹, —S(═O)₂NR⁹R¹⁰ and R⁹.

7. The compound of claim 1 wherein R is selected from the group consisting of —NR⁶R⁷, —NO₂, F, Cl, Br, I, —CN, C₁-C₆ alkyl, C₆-C₁₀ aryl, and O—(C₆-C₁₀) aryl.

8. The compound of claim 1 wherein R¹ is (C₁-C₈) alkyl, and R² is selected from the group consisting of F, Cl, Br, I, nitro, —CN, CF₃, (C₁-C₈)alkyl, (C₆-C₁₀) aryl, and 5-12 membered heteroaryl, —NR⁶R⁷, OR⁶ wherein each said alkyl, cycloalkyl, heterocycloalkyl, aryl and heteroaryl is optionally substituted with one or more substituents independently selected from F, Cl, Br, I, nitro, CN, CF₃, —NR⁹R¹⁰, —NR⁹C(═O)R¹⁰, —OR⁹, —C(═O)OR⁹, —C(═O)R⁹, —C(═O)NR⁹R¹⁰, —SR⁹, —S(═O)R⁹, —S(═O)₂R⁹, —S(═O)₂NR⁹R¹⁰ and R⁹.

9. The compound of claim 1 wherein R¹ is (C₁-C₈) alkyl, and R² is selected from the group consisting of —NR⁶R⁷, —NO₂, F, Cl, Br, I, —CN, (C₁-C₆) alkyl, (C₆-C₁₀) aryl, and O—(C₆-C₁₀) aryl.

10. The compound of claim 1 wherein R¹ is (C₁-C₈) alkyl, and R² is selected from the group consisting of —NR⁶R⁷, —NO₂, F, Cl, Br, I, —CN, (C₁-C₆) alkyl, phenyl, and O-phenyl.

11. The compound of claim 1 wherein R¹ is (C₆-C₁₀) aryl, and R² is selected from the group consisting of F, Cl, Br, I, nitro, —CN, CF₃, (C₁-C₈)alkyl, (C₆-C₁₀) aryl, and 5-12 membered heteroaryl, —NR⁶R⁷, OR⁶ wherein each said alkyl, cycloalkyl, heterocycloalkyl, aryl and heteroaryl is optionally substituted with one or more substituents independently selected from F, Cl, Br, I, nitro, CN, CF₃, —NR⁹R¹⁰, —NR⁹C(═O)R¹⁰, —OR⁹, —C(═O)OR⁹, —C(═O)R⁹, —C(═O)NR⁹R¹⁰, —SR⁹, —S(═O)R⁹, —S(═O)₂R⁹, —S(═O)₂NR⁹R¹⁰ and R⁹.

12. The compound of claim 1 wherein R¹ is (C₆-C₁₀) aryl, and R² is selected from the group consisting of —NR⁶R⁷, —NO₂, F, Cl, Br, I, —CN, (C₁-C₆) alkyl, and O—(C₆-C₁₀) aryl.

13. A compound of claim 1 selected from the group consisting of:
4-(6-bromo-5-methyloxazolo[4,5-b]pyridin-2-yl)-1,4-diaza-bicyclo[3.2.2]nonane
4-(6-Bromo-5-ethyl-oxazolo[4,5-b]pyridin-2-yl)-1,4-diaza-bicyclo[3.2.2]nonane
4-(5,6-dimethyloxazolo[4,5-b]pyridin-2-yl)-1,4-diaza-bicyclo[3.2.2]nonane
4-(6-Methyl-5-ethyl-oxazolo[4,5-b]pyridin-2-yl)-1,4-diaza-bicyclo[3.2.2]nonane
4-(5-Methyl-6-nitro-oxazolo[4,5-b]pyridin-2-yl)-1,4-diaza-bicyclo[3.2.2]nonane
2-(1,4-Diaza-bicyclo[3.2.2]non-4-yl)-5-methyl-oxazolo[4,5-b]pyridin-6-ylamine
4-(6-Fluoro-5-methyl-oxazolo[4,5-b]pyridin-2-yl)-1,4-diaza-bicyclo[3.2.2]nonane
4-(6-Chloro-5-methyl-oxazolo[4,5-b]pyridin-2-yl)-1,4-diaza-bicyclo[3.2.2]nonane
4-(6-Chloro-5-ethyl-oxazolo[4,5-b]pyridin-2-yl)-1,4-diaza-bicyclo[3.2.2]nonane
4-(5-methyl-6-phenyloxazolo[4,5-b]pyridin-2-yl)-1,4-diaza-bicyclo[3.2.2]nonane
4-(5-methyl-6-phenoxyoxazolo[4,5-b]pyridin-2-yl)-1,4-diaza-bicyclo[3.2.2]nonane
2-(1,4-diaza-bicyclo[3.2.2]nonan-4-yl)-5-methyloxazolo[4,5-b]pyridine-6-carbonitrile
or a pharmaceutically acceptable salt, hydrate, or solvate thereof.

14. A pharmaceutical composition comprising a compound according to claim 1, or a pharmaceutically acceptable thereof, and a pharmaceutically acceptable carrier.

15. A method for treating schizophrenia in a mammal, comprising administering to said mammal an amount of a compound according to claim 1, or a pharmaceutically acceptable salt thereof, that is effective in treating schizophrenia.

16. A method for treating a disorder or condition selected from cognitive and attention deficit symptoms of Alzheimer's, neurodegeneration associated with Alzheimer's disease, pre-senile dementia (mild cognitive impairment), or senile dementia, schizophrenia, cognitive deficits associated with schizophrenia, psychosis, cognitive deficits associated with psychosis, attention deficit disorder, attention deficit hyperactivity disorder (ADHD), mood and affective disorders, amyotrophic lateral sclerosis, borderline personality disorder, traumatic brain injury, behavioral and cognitive problems associated with brain tumors, AIDS dementia complex, dementia associated with Down's syndrome, dementia associated with Lewy Bodies, Huntington's disease, depression, general anxiety disorder, age-related macular degeneration, Parkinson's disease, tardive dyskinesia, Pick's disease, post traumatic stress disorder, dysregulation of food intake including bulemia and anorexia nervosa, withdrawal symptoms associated with smoking cessation and dependent drug cessation, Tourette's syndrome, glaucoma, neurodegeneration associated with glaucoma, symptoms associated with pain, pain and inflammation, TNF-α related conditions, rheumatoid arthritis, rheumatoid spondylitis, muscle degeneration, osteoporosis, osteoarthritis, psoriasis, contact dermatitis, bone resorption diseases, atherosclerosis, Paget's disease, uveititis, gouty arthritis, inflammatory bowel disease, adult respiratory distress syndrome (ARDS), Crohn's disease, rhinitis, ulcerative colitis, anaphylaxis, asthma, Reiter's syndrome, tissue rejection of a graft, ischemia reperfusion injury, stroke, multiple sclerosis, cerebral malaria, sepsis, septic shock, toxic shock syndrome, fever and myalgias due to infection, HIV-1, HIV-2, and HIV-3, cytomegalovirus (CMV), influenza, adenovirus, a herpes virus (including HSV-1, HSV-2), a herpes zoster, cancer (multiple myeloma, acute and chronic myelogenous leukemia, or cancer-associated cachexia), diabetes (pancreatic beta cell destruction, or type I and type II diabetes), wound healing (healing burns, and wounds in general including from surgery), bone fracture healing, ischemic heart disease, tinnitus, or stable angina pectoris in a mammal, comprising administering to a mammal in need of such treatment an amount of a compound according to claim 1, or a pharmaceutically acceptable salt thereof, that is effective in treating such disorder or condition.

17. A method for treating a disease or condition in a mammal in need of treatment, wherein the mammal receives symptomatic relief from activation of an α7 nicotinic acetylcholine receptor, comprising administering to a mammal in need of such treatment a compound of the formula I, or a pharmaceutically acceptable salt thereof.

18. The method of claim 17, wherein the disease or condition is selected from cognitive and attention deficit symptoms of Alzheimer's Disease, neurodegeneration associated with Alzheimer's Disease, pre-senile dementia (mild cognitive impairment), or senile dementia, schizophrenia, cognitive deficits associated with schizophrenia, psychosis, cognitive deficits associated with psychosis, attention deficit disorder, attention deficit hyperactivity disorder, mood and affective disorders, amyotrophic lateral sclerosis, borderline personality disorder, traumatic brain injury, behavioral and cognitive problems associated with brain tumors, AIDS dementia complex, dementia associated with Down's syndrome, dementia associated with Lewy Bodies, Huntington's disease, depression, general anxiety disorder, age-related macular degeneration, Parkinson's disease, tardive dyskinesia, Pick's disease, post traumatic stress disorder, dysregulation of food intake including bulemia and anorexia nervosa, withdrawal symptoms associated with smoking cessation and dependent drug cessation, Gilles de la Tourette's Syndrome, glaucoma, neurodegeneration associated with glaucoma, or symptoms associated with pain.

19. The method of claim 18, wherein the disease or condition is selected from cognitive deficits associated with schizophrenia, cognitive and attention deficit symptoms of Alzheimer's Disease, and neurodegeneration associated with Alzheimer's Disease.

20. A pharmaceutical composition comprising a compound of claim 1, or a pharmaceutically acceptable salt thereof, and an antipsychotic drug or a pharmaceutically acceptable salt thereof.

21. The pharmaceutical composition of claim 20, wherein the antipsychotic drug or pharmaceutically acceptable salt thereof is selected from the group consisting of Chlorpromazine, Fluphenazine, Haloperidol, Loxapine, Mesoridazine, Molindone, Perphenazine, Pimozide, Thioridazine, Thiothixene, or Trifluoperazine, Asenapine, Ziprasidone, Olanzapine, Clozapine, Risperidone, Sertindole, Quetiapine, Aripiprazole, Amisulpride, Paliperidone, and Bifeprunox, and pharmaceutically acceptable salts thereof.

22. The pharmaceutical composition of claim 21, wherein the antipsychotic drug or salt is Ziprasidone or a pharmaceutically acceptable salt thereof.

23. A method of treating a mammal suffering from cognitive impairment, schizophrenia or psychosis, comprising administering to said mammal a compound of claim 1, or a pharmaceutically acceptable salt thereof, and an antipsychotic drug or pharmaceutically acceptable salt thereof, wherein the amounts of each together are effective in treating the cognitive impairment, schizophrenia or psychosis.

24. A method of treating a male mammal suffering from infertility, comprising administering a compound of claim 1, or a pharmaceutically acceptable salt thereof, in an amount effective in treating the infertility.

25. The method of claim 16, wherein the disorder or condition is inflammation.
